(19) United States
(12) Patent Application Publication
(10) Pub. No.: US 2017/0283352 A1
(43) Pub. Date: Oct. 5, 2017

(54) METHOD FOR PRODUCING AN AROMA SUBSTANCE

(71) Applicant: BASF SE, Ludwigshafen (DE)

(72) Inventors: Thomas FENLON, Mannheim (DE); Sumana CHATURVEDULA, Mannheim (DE); Dominic RIEDEL, Lampertheim (DE); Stefan RÜDENAUER, Weinheim (DE); Ralf PELZER, Fürstenberg (DE); Pepa DIMITROVA, Worms (DE); Florina Corina PATCAS, Ludwigshafen (DE); Manuel DANZ, Plankstadt (DE)

(21) Appl. No.: 15/509,238

(22) PCT Filed: Sep. 8, 2015
(86) PCT No.: PCT/EP2015/070492

§ 371 Date: Mar. 7, 2017

(30) Foreign Application Priority Data
Sep. 9, 2014 (EP) 14184119.7

(51) Int. Cl. C07C 45/54; C07C 45/80

(52) U.S. Cl. CPC C07C 45/54; C07C 45/80

(57) ABSTRACT

A method of preparing a compound of formula (IV)

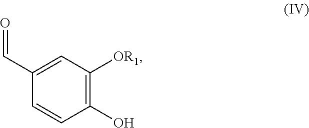

where R₁ is alkyl of 1 to 4 carbon atoms, comprises reacting cyclohexene with hydrogen peroxide and an alcohol R₁OH in the presence of a catalyst comprising a zeolite of framework structure MWW, wherein the framework of the zeolite comprises silicon, titanium, boron, oxygen and hydrogen.

The present invention relates to a method of preparing a compound of the formula (IV)

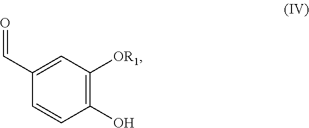

wherein R₁ is alkyl of 1 to 4 carbon atoms, and where an intermediate stage, the compound of formula (I)

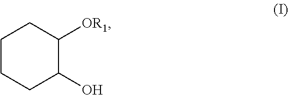

is prepared from cyclohexene by use of hydrogen peroxide and of a catalyst comprising a zeolite of framework structure MWW, wherein the framework of the zeolite as per (ii) comprises silicon, titanium, boron, oxygen and hydrogen. The present invention more particularly relates to a method of preparing vanillin via guaiacol as intermediate stage.

Vanillin (3-methoxy-4-hydroxybenzaldehyde) or ethylvanillin, but also isopropylvanillin, as examples of a compound of formula (IV) are some of the most important aroma chemicals in the world. Vanillin is the number two food additive, behind aspartame. The main field of use for vanillin is in the food industry, although the artificial flavoring substance ethylvanillin is increasingly being used as a substitute for vanillin. Vanillin also finds use as a fragrance in the perfume industry and as an intermediate in the pharmaceutical industry. Vanillin is derivable from natural sources such as, for instance, lignin or ferulic acid, although a significant proportion is obtained synthetically. The majority of these syntheses proceed via the intermediate known as guaiacol (2-methoxyphenol).

There is accordingly a constant need for economically advantageous methods of synthesis to prepare compounds of formula (IV) such as, for example, vanillin or ethylvanillin, particularly via the compound of formula (I), for example 2-methoxyphenol or 2-ethoxyphenol, as an intermediate stage.

GB 2 252 556 A discloses a method of preparing 2-methoxy- and 2-ethoxycyclohexanols by reacting cyclohexene with hydrogen peroxide, methanol or, respectively, ethanol and optionally sulfuric acid in the presence of a catalyst composition prepared by drying and calcining a mixture of titanium tetraethoxide and silica gel in hexane or ethanol. Only when employed to prepare 2-methoxycyclohexanol does this method achieve a target product selectivity, for 2-methoxycyclohexanol, of 95%, albeit with the disadvantage that the admixture of sulfuric acid to the reaction mixture is required to achieve this high selectivity.

A. Corma et al., “Activity of Ti-Beta Catalyst for the Selective Oxidation of Alkenes and Alkanes”, Journal of Catalysis (1994), volume 145, pages 151-158 discloses a method of preparing 2-alkoxycyclohexanol by reacting cyclohexene with hydrogen peroxide and an alkyl alcohol selected from the group consisting of methanol, ethanol and tert-butanol in the presence of a catalyst in the form of a Ti—Al-Beta zeolite. This method has the disadvantage that high excesses based on hydrogen peroxide are employed of cyclohexene and alkyl alcohol.

Y. Goa et al., “Catalytic Performance of [Ti, Al]-Beta in the Alkene Epoxidation Controlled by the Postsynthetic ion Exchange”, Journal of Physical Chemistry B 2004, volume 108, pages 8401-8411 discloses a method of preparing 2-methoxycyclohexanol by reacting cyclohexene with hydrogen peroxide and methanol in the presence of a catalyst in the form of a Ti—Al-Beta zeolite. The ostensibly high selectivities reported for this method are based not on the target product, 2-methoxycyclohexanol, but on a mixture consisting of 1,2-cyclohexanediol and 2-methoxycyclohexanol.

E. G. Derouane et al., “Titanium-substituted zeolite beta: an efficient catalyst in the oxy-functionalisation of cyclic alkenes using hydrogen peroxide in organic solvents”, New. J. Chem., 1998, pages 797-799 discloses a method of preparing 2-alkoxycyclohexanol by reacting cyclohexene with hydrogen peroxide and an alkyl alcohol selected from the group consisting of methanol, ethanol, propanol, isopropyl alcohol and tert-butanol in the presence of a catalyst in the form of a Ti—Al-Beta zeolite.

WO 2014/016146 A1 describes a method of preparing vanillin from 1,2-dihydroxybenzene.

CN 103 709 018 A describes the preparation of guaiacol from cyclohexene oxide by reaction with methanol and subsequent dehydrogenation.

It is an object of the present invention to provide a novel method of preparing compounds of general formula (IV)

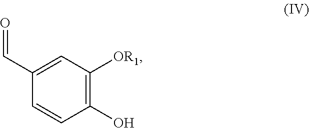

wherein R₁ is alkyl of 1 to 4 carbon atoms.

It is more particularly an object of the present invention to provide a novel method of preparing compounds of general formula (IV)

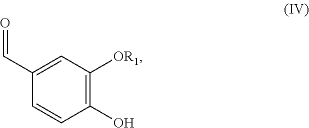

wherein R₁ is alkyl of 1 to 4 carbon atoms, via the intermediate stage of formula (I)

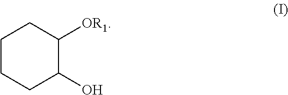

We have found that, surprisingly, this object is achieved when the intermediate stage, the compound of formula (I)

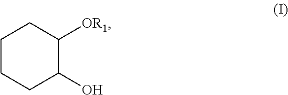

is prepared by employing a specific zeolitic material of framework structure MWW as a catalyst having a high selectivity for the target product, the compound of formula (I).

The present invention accordingly provides a method of preparing a compound of formula (IV)

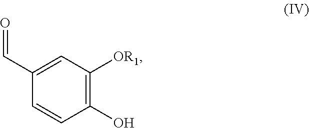

wherein R₁ is alkyl of 1 to 4 carbon atoms, comprising
(i) providing a liquid mixture comprising cyclohexene, an alcohol R₁OH, hydrogen peroxide and optionally a solvent;
(ii) reacting the cyclohexene with the hydrogen peroxide and the alcohol R₁OH in the mixture provided as per (i) in the presence of a catalyst comprising a zeolite of framework structure MWW to obtain a mixture comprising the compound of formula (I)

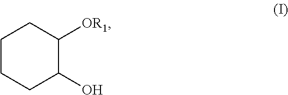

wherein the framework of the zeolite as per (ii) comprises silicon, titanium, boron, oxygen and hydrogen;
(iii) separating the compound of formula (I) off from the mixture obtained as per (ii) to obtain a mixture concentrated in respect of the compound of formula (I);
(iv) dehydrogenating the formula (I) compound present in the concentrated mixture obtained as per (iii) to obtain a mixture comprising a compound of formula (II)

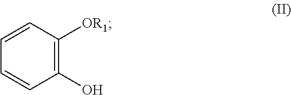

(v) formylating the formula (II) compound present in the mixture obtained as per (iv) to obtain a mixture comprising the compound of formula (IV).

### Step (i)

R₁ in the compound of formula (I) and the alcohol R₁OH is alkyl of 1 to 4 carbon atoms, i.e., of 1, 2, 3 or 4 carbon atoms. Step (i) permits the use of a mixture of two or more alcohols R₁OH that differ in alkyl R₁. R₁ may in principle be suitably substituted, in which case R₁ may have one or more substituents, which may each be, for example, hydroxyl, chloro, fluoro, bromo, iodo, nitro or amino. Alkyl R₁ is preferably unsubstituted alkyl, preferably selected from the group consisting of methyl, ethyl, n-propyl, isopropyl, n-butyl, isobutyl and tert-butyl, more preferably from the group consisting of methyl, ethyl, n-propyl, and isopropyl, more preferably from the group consisting of methyl and ethyl. R₁ is more preferably methyl.

The composition of the liquid mixture as per (i) is in principle not subject to any special restriction.

In principle, the liquid mixture provided as per (i) may have a molar ratio prior to the reaction as per (ii) of cyclohexene:R₁OH less than, equal to or greater than 1:1. Preferably, the liquid mixture provided as per (i) has a molar ratio prior to the reaction as per (ii) of cyclohexene:R₁OH not more than 1:1. Further preferably the liquid mixture provided as per (i) has a molar ratio prior to the reaction as per (ii) of cyclohexene:R₁OH in the range from 1:1 to 1:50, more preferably from 1:3 to 1:30 and more preferably from 1:5 to 1:10.

In principle, the liquid mixture provided as per (i) may comprise a solvent. Any solvent is preferably selected from the group consisting of C1-C6-alkyl nitriles, i.e., C1-, C2-, C3-, C4-, C5- or C6-alkyl nitriles, dialkyl ketones of the formula R₂—CO—R₃, where R₂ and R₃ are each independently selected from the group consisting of C1-C6-alkyl, i.e., C1-, C2-, C3-, C4-, C5- or C6-alkyl, and a mixture of two or more thereof, more preferably selected from the group consisting of C1-C3-alkyl nitriles, i.e., C1-, C2- or C3-alkyl nitriles, dialkyl ketones of the formula R₂—CO—R₃, where R₂ and R₃ are each independently selected from the group consisting of C1-C3-alkyl, i.e., C1-, C2- or C3-alkyl, and a mixture of two or more thereof, more preferably selected from the group consisting of acetonitrile, acetone and a mixture thereof.

When a solvent is included, the molar ratio of solvent:cyclohexene in the liquid mixture provided as per (i) may in principle be less than, equal to or greater than 1:1 prior to the reaction of (ii). Preferably, the molar ratio of solvent:cyclohexene in the liquid mixture provided as per (i) is not less than 1:1 before the reaction of (ii). More preferably, the molar ratio of solvent:cyclohexene in the liquid mixture provided as per (i) is in the range from 20:1 to 1:1, more preferably from 15:1 to 1:1, more preferably from 10:1 to 1:1, before the reaction of (ii). When the solvent in the mixture comprises a mixture of two or more solvents, the molar ratio of solvent:cyclohexene is based on the mixture of solvents.

The mixture provided as per (i) preferably comprises no solvent. In this case, the liquid mixture provided as per (i) is preferably not less than 90 wt %, more preferably not less than 95 wt %, more preferably not less than 98 wt %, more preferably not less than 99 wt %, more preferably not less than 99.5 wt %, more preferably not less than 99.9 wt % comprised of cyclohexene, R₁OH, methanol, hydrogen peroxide and any water from hydrogen peroxide being employed, as described below, in the form of an aqueous solution.

The temperature at which the liquid mixture is provided as per (i) is in principle not subject to any restriction. The liquid mixture as per (i) is preferably provided at a temperature in the range from 5 to 50° C., more preferably at a temperature in the range from 10 to 40° C., more preferably at a temperature in the range from 15 to 30° C.

The step of providing the liquid mixture as per (i) is in principle not subject to any special restriction. For instance, the liquid mixture as per (i) is providable by mixing the cyclohexene, the alcohol R₁OH, the hydrogen peroxide and optionally the solvent in any order. The liquid mixture as per (i) is preferably provided by admixing the hydrogen peroxide to a mixture comprising the cyclohexene, the alcohol R₁OH and optionally the solvent. It is preferable in this case for the mixture comprising the cyclohexene, the alcohol R₁OH and the optional solvent to be presented as the initial charge at a temperature in the range from 5 to 50° C., more preferably from 10 to 40° C., more preferably from 15 to 30° C., and for the temperature of the mixture resulting from admixing the hydrogen peroxide to be suitably maintained within the aforementioned temperature ranges.

The hydrogen peroxide is preferably admixed in the form of a solution in one or more suitable solvents. Possible solvents include, for example, water or organic solvents such as, for example, organic solvents selected from the group consisting of C1-C6-alcohols, C1-C6-alkyl nitriles, dialkyl ketones of the formula R₂—CO—R₃, where R₂ and R₃ are each independently selected from the group consisting of C1-C6-alkyl, and a mixture of two or more thereof, preferably from the group consisting of C1-C3-alcohols, C1-C3-alkyl nitriles, dialkyl ketones of the formula R₂—CO—R₃, where R₂ and R₃ are each independently selected from the group consisting of C1-C3-alkyl, and a mixture of two or more thereof, more preferably from the group consisting of methanol, acetonitrile, acetone and a mixture thereof. The hydrogen peroxide is preferably admixed in the form of a methanolic or aqueous, preferably aqueous, solution. The hydrogen peroxide content of the preferably aqueous solution is not subject to any special restrictions and preferably ranges from 25 to 75 wt %, more preferably from 40 to 70 wt %, based on the overall weight of the aqueous solution.

In principle, the molar ratio of cyclohexene:hydrogen peroxide in the liquid mixture provided as per (i) may be less than, equal to or greater than 1:1 before the reaction of (ii). Preferably, the molar ratio of cyclohexene:hydrogen peroxide in the liquid mixture provided as per (i) is not less than 1:1 before the reaction of (ii). More preferably, the molar ratio of cyclohexene:hydrogen peroxide in the liquid mixture provided as per (i) is in the range from 1:1 to 5:1, more preferably from 1.5:1 to 4.5:1, more preferably from 2:1 to 4:1, before the reaction of (ii).

The mixture provided as per (i) preferably comprises no strong nonnucleophilic inorganic acid, preferably no sulfuric acid.

### Step (ii)

### Catalyst

The inventors found that, surprisingly, the catalyst employed in (ii) has a high level of selectivity for the target product 2-alkoxycyclohexanol. The catalyst employed as per (ii) is not subject to any special restrictions. Preferably, however, the zeolitic material of framework structure MWW evinces one or more of the following features as per the itemized embodiments, including the combinations of embodiments as per the stated dependencies:
1. The zeolitic material of framework structure MWW wherein the framework of the zeolitic material comprises boron and titanium, wherein preferably not less than 99 wt %, more preferably not less than 99.5 wt %, more preferably not less than 99.9 wt % of the framework of the zeolitic material consists of silicon, titanium, boron, oxygen and hydrogen.
2. The zeolitic material according to Embodiment 1 wherein the molar ratio of B:Si is in the range from 0.02:1 to 0.5:1, preferably from 0.05:1 to 0.15:1 and the molar ratio of Ti:Si is in the range from 0.01:1 to 0.05:1, preferably from 0.017:1 to 0.025:1.
3. The zeolitic material according to Embodiment 1 or 2 wherein the zeolitic material is present in the calcined state.
4. The zeolitic material according to Embodiment 3 wherein the calcined state of the zeolitic material is obtained by subjecting the zeolitic material in its non-calcined state to a calcination at a temperature in the range from 500 to 700° C., preferably from 550 to 700° C., more preferably from 600 to 700° C., preferably for a time period in the range from 0.1 to 24 h, more preferably from 1 to 18 h, more preferably from 6 to 12 h, preferably in an atmosphere comprising oxygen.
5. The zeolitic material according to any one of Embodiments 1 to 4 with an MWW-templating compound content of not more than 0.5 wt %, preferably of not more than 0.2 wt %, more preferably of not more than 0.1 wt %, based on the overall weight of the zeolite and reckoned as total organic carbon (TOC) content of the calcined zeolitic material.
6. The zeolitic material according to any one of Embodiments 1 to 5 wherein the ²⁹Si NMR spectrum of the zeolitic material comprises:
   a first signal in the range from −95.0 to −105.0 ppm,
   a second signal in the range from −105.0 to −115.0 ppm,
   a third signal in the range from −115.0 to −125.0 ppm,
   wherein the ratio of the integral of the range of the first signal to the integral of the range of the third signal is preferably in the range from 0.6 to 1.1, more preferably in the range from 0.7 to 1.0, more preferably in the range from 0.8 to 0.9.
7. The zeolitic material according to any one of Embodiments 1 to 6 wherein the ¹¹B NMR spectrum of the zeolitic material comprises:
   a first signal in the range from 20.0 to 10.0 ppm,
   a second signal in the range from 10.0 to 1.0 ppm, preferably having a peak in the range from 6.5 to 5.5 ppm, more preferably from 6.2 to 5.8 ppm,
   a third signal in the range from 1.0 to −7.0 ppm, preferably having a peak in the range from −2.4 to −3.4 ppm, more preferably from −2.7 to −3.1 ppm,
   a fourth signal in the range from −7.0 to −16.0 ppm,
   wherein the ratio of the integral of the range of the third signal to the integral of the range of the second signal is preferably in the range from 1.00 to 1.15, more preferably in the range from 1.05 to 1.15, more preferably in the range from 1.10 to 1.15.
8. The zeolitic material according to any one of Embodiments 1 to 7 with a water uptake in the range from 12.0 to 16.0 wt %, preferably from 12.0 to 15 wt %, more preferably from 12 to 14 wt %.
9. The zeolitic material according to any one of Embodiments 1 to 8 with a specific surface area (BET) in the range from 400 to 500 m²/g, preferably from 410 to 490 m²/g, more preferably from 420 to 480 m²/g, as determined to DIN 66131.
10. The zeolitic material according to any one of Embodiments 1 to 9 wherein the infrared spectrum of the zeolitic material comprises a band at (3748±20) cm⁻¹, a band at (3719±20) cm⁻¹, a band at (3689±20) cm⁻¹, a band at (3623±20) cm⁻¹, a band at (3601±20) cm⁻¹ and a band at (3536±20) cm⁻¹.
11. The zeolitic material according to any one of Embodiments 1 to 10 characterized by an x-ray diffractogram having peaks at 2 theta angles of (7.2±0.1)°, (14.5±0.1)°, (22.1±0.1°), (22.7±0.1)°, (23.0±0.1°), (24.0±0.1)°, (25.3±0.1)°, (26.3±0.1)°, (27.3±0.1)°, (28.1±0.1°).
12. The zeolitic material according to Embodiment 11, characterized by an x-ray diffractogram further comprising peaks at 2 theta angles of (7.0±0.1°), (8.1±0.1)°, (10.1±0.1)°, (14.3±0.1°), (20.4±0.1°), (21.9±0.1)°, (28.9±0.1°), (33.8±0.1°), (47.0±0.1°), (65.4+0.1)°, (66.4±0.1)°.
13. The zeolitic material according to any one of Embodiments 1 to 12 with a boron content, reckoned as elemental boron, in the range from 0.9 to 2.2 wt %, preferably from 1.0 to 2.0 wt %, more preferably from 1.1 to 1.8 wt %, more preferably from 1.2 to 1.6 wt %, based on the overall weight of the zeolitic material.
14. The zeolitic material according to any one of Embodiments 1 to 13 with a titanium content, reckoned as elemental titanium, in the range from 0.9 to 3 wt %, preferably from 1.0 to 2.0 wt %, more preferably from 1.1 to 1.8 wt %, more preferably from 1.2 to 1.6 wt %, based on the overall weight of the zeolitic material.
15. The zeolitic material according to any one of Embodiments 1 to 14 with a boron content, reckoned as elemental boron, in the range from 1.2 to 1.5 wt % and a titanium content, reckoned as elemental titanium, in the range from 1.2 to 1.5 wt %, based on the overall weight of the zeolitic material.
16. The zeolitic material according to any one of Embodiments 1 to 15 in a molding,
17. The zeolitic material according to Embodiment 16 wherein the molding further comprises at least one binder material, preferably silicon dioxide.
18. The zeolitic material according to any one of Embodiments 1 to 17, obtainable or obtained as per a method comprising
   (a) providing an aqueous synthesis mixture comprising a silicon source, a boron source, a titanium source and an MWW-templating compound, wherein the temperature of the aqueous synthesis mixture is not more than 50° C.;
   (b) heating the aqueous synthesis mixture provided as per (a) from the temperature of not more than 50° C. to a temperature in the range from 160 to 190° C. in the course of a period of at most 24 h;
   (c) subjecting the synthesis mixture as per (b) to hydrothermal synthesis conditions under autogenous pressure in a closed system at a temperature in the range from 160 to 190° C. to obtain a precursor to the zeolite of framework structure MWW in its mother liquor;
   (d) separating the precursor to the zeolite of framework structure MWW off from its mother liquor;
   (e) calcining the MWW framework structure zeolite precursor separated off as per (d) to obtain the zeolite of framework structure MWW.
19. The zeolitic material according to Embodiment 18 wherein the aqueous synthesis mixture provided as per (a) is obtained by admixing the silicon source to an aqueous mixture comprising the boron source, the titanium source and the MWW-templating compound.
20. The zeolitic material according to Embodiment 18 or 19 wherein the aqueous mixture comprising the boron source, the titanium source and the MWW-templating compound is obtained by admixing a mixture comprising the silicon source and some of the MWW-templating compound to an aqueous mixture comprising the boron source and some of the MWW-templating compound, wherein the mixture comprising some of the MWW-templating compound and some of the titanium source preferably comprises no water.
21. The zeolitic material according to any one of Embodiments 18 to 20 wherein the aqueous synthesis mixture after the step of admixing the silicon source is stirred at a temperature of not more than 50° C. for a time period in the range from 45 to 180 min, preferably from 60 to 120 min, more preferably from 80 to 100 min.
22. The zeolitic material according to any one of Embodiments 18 to 21 wherein as per (a) the silicon source is selected from the group consisting of fumed silica, colloidal silica, silicon alkoxides and a mixture of two or more thereof, preferably from the group consisting of fumed silica, colloidal silica and a mixture thereof, wherein the silicon source is more preferably fumed silica;
   the boron source is selected from the group consisting of boric acid, borates, boron oxide and a mixture of two or more thereof, preferably from the group consisting of boric acid, borates and a mixture thereof, wherein the boron source is more preferably boric acid; the titanium source is selected from the group consisting of titanium alkoxides, titanium halides, titanium salts, titanium dioxide and a mixture of two or more thereof, preferably from the group consisting of titanium alkoxides, titanium halides and a mixture thereof, wherein the titanium source is more preferably a titanium alkoxide, more preferably titanium tetrabutoxide;
   the MWW-templating compound is selected from the group consisting of piperidine, hexamethyleneimine, N,N,N,N′,N′,N′-hexamethyl-1,5-pentanediammonium salts, 1,4-bis(N-methylpyrrolidinyl)butane, octyltrimethylammonium hydroxide, heptyltrimethyl-ammonium hydroxide, hexyltrimethylammonium hydroxide and a mixture of two or more thereof, preferably from the group consisting of piperidine, hexamethyleneimine and a mixture thereof, wherein the MWW-templating compound is more preferably piperidine.
23. The zeolitic material according to any one of Embodiments 18 to 22 wherein the aqueous synthesis mixture comprises:
   the boron source, reckoned as elemental boron, based on the silicon source, reckoned as elemental silicon, in a molar ratio ranging from 0.18:1 to 5.2:1, preferably from 0.5:1 to 3:1; the titanium source, reckoned as elemental titanium, based on the silicon source, reckoned as elemental silicon, in a molar ratio ranging from 0.005:1 to 0.15:1, preferably from 0.01:1 to 0.1:1;
   the MWW-templating compound based on the silicon source, reckoned as elemental silicon, in a molar ratio ranging from 0.4:1 to 4.2:1, preferably from 0.6:1 to 2:1; the water, based on the silicon source, reckoned as elemental silicon, in a molar ratio ranging from 1:1 to 30:1, preferably from 2:1 to 25:1.
24. The zeolitic material according to any one of Embodiments 18 to 23 wherein the aqueous synthesis mixture provided as per (a) has a pH in the range from 10 to 13, preferably from 10.5 to 12.5, more preferably from 11 to 12, as determined using a pH-sensitive glass electrode.
25. The zeolitic material according to any one of Embodiments 18 to 24 wherein step (b) of heating the aqueous synthesis mixture provided as per (a) from the temperature of not more than 50° C. to a temperature in the range from 160 to 190° C. is effected within a time period ranging from 2 to 18 h, preferably from 4 to 14 h, more preferably from 8 to 12 h, preferably under agitation.
26. The zeolitic material according to any one of Embodiments 18 to 25 wherein step (b) of heating the mixture provided as per (a) from the temperature of not more than 50° C. to a temperature in the range from 160 to 190° C. is carried out in a continuous manner.
27. The zeolitic material according to any one of Embodiments 18 to 26 wherein step (c) of subjecting the synthesis mixture to hydrothermal synthesis conditions under autogenous pressure is effected for a time period in the range from 80 to 200 h, preferably from 100 to 180 h, more preferably from 120 to 160 h, preferably under at least temporary agitation.
28. The zeolitic material according to any one of Embodiments 18 to 27 wherein step (d) of separating off comprises
   (d.1) washing the precursor to the zeolitic material of framework structure MWW, preferably with water, until the water used for said washing has a pH of less than 10, as determined using a pH-sensitive glass electrode;
   (d.2) drying the washed precursor to the zeolitic material of framework structure MWW, preferably at a temperature in the range from 10 to 150° C., more preferably at a temperature in the range from 30 to 130° C., preferably in an atmosphere comprising oxygen.
29. The zeolitic material according to any one of Embodiments 18 to 28 wherein the precursor to the zeolitic material of framework structure MWW is treated neither before nor during (d) with an aqueous solution having a pH of not more than 6, as determined using a pH-sensitive glass electrode.
30. The zeolitic material according to any one of Embodiments 18 to 29 wherein step (e) of calcining is carried out at a temperature in the range from 500 to 700° C., preferably from 550 to 700° C., more preferably from 600 to 700° C., preferably for a time period in the range from 0.1 to 24 h, more preferably from 1 to 18 h, more preferably from 6 to 12 h.
31. The zeolitic material according to any one of Embodiments 18 to 30 wherein the MWW framework structure zeolitic material precursor obtained from (c) is not treated before (d) with an aqueous solution having a pH of not more than 6, as determined using a pH-sensitive glass electrode, and wherein the calcined zeolitic material of framework structure MWW is not treated after (d) with an aqueous solution having a pH of not more than 6, as determined using a pH-sensitive glass electrode.
32. The zeolitic material according to any one of Embodiments 18 to 31 wherein the boron content of the precursor to the MWW framework structure zeolitic material which is subjected to a calcination is not less than 90%, preferably not less than 95%, more preferably not less than 98% of the boron content of the precursor obtained from the hydrothermal synthesis as per (c).
33. The zeolitic material according to any one of Embodiments 18 to 32 wherein the boron content of the MWW framework structure zeolitic material obtained from the calcining step as per (d) is not less than 90%, preferably not less than 95%, more preferably not less than 98% of the boron content of the precursor obtained from the hydrothermal synthesis as per (c).
34. The zeolitic material according to any one of Embodiments 18 to 33 wherein the method further comprises
   (f) molding the MWW framework structure zeolitic material obtained from (d) or (e) to obtain a molding;
   (g) optionally drying and/or calcining the molding obtained from (f).
35. The zeolitic material according to any one of Embodiments 18 to 34 wherein the MWW framework structure zeolitic material is not before (f) treated with an aqueous solution having a pH of not more than 6, as determined using a pH-sensitive glass electrode, and wherein the molding comprising the zeolitic material of framework structure MWW is not after steps (f) and (g) treated with an aqueous solution having a pH of not more than 6, as determined using a pH-sensitive glass electrode.
36. The zeolitic material according to Embodiment 34 or 35 wherein the boron content of the MWW framework structure zeolitic material in the molding obtained as per (f) or (g), preferably as per (g), is not less than 90%, preferably not less than 95%, more preferably not less than 98% of the boron content of the precursor obtained from the hydrothermal synthesis as per (c).
37. A molding comprising a zeolitic material according to any one of Embodiments 1 to 15 or 18 to 36.

### Reaction as Per Step (ii)

As far as the amount of catalyst is concerned, the mass ratio of hydrogen peroxide:zeolitic material of framework structure MWW is preferably in the range from 10:1 to 0.1:1, preferably from 1:1 to 0.2:1, more preferably from 0.75:1 to 0.25:1, at the start of the reaction as per (ii).

The reaction as per (ii) may generally be carried out as per any suitable procedure. Options thus include, for example, a discontinuous process in one or more batch reactors or a continuous process in one or more reactors operated in a continuous manner and optionally interconnected in series and/or in parallel.

The procedure for performing the reaction as per (ii) in a discontinuous process is not subject to any special restriction. A suitable reactor for the reaction as per (ii) is, for example, a reactor fitted with suitable heating means, a suitable stirrer and a reflux condenser. The reaction as per (ii) is preferably carried out in an open system. The reaction as per (ii) is preferably carried out under suitable agitation of the reaction mixture, for example stirring, in which case the energy input due to agitation may be varied or kept substantially constant during the reaction. The energy input may be suitably chosen according to, for example, the volume of the reaction mixture, the form of the catalyst or the reaction temperature. When the reaction as per (ii) is carried out in batch mode, the catalyst used as per (ii) is preferably an MWW framework structure zeolitic material as described above in Embodiments 1 to 15 and 18 to 33.

The procedure for performing the reaction as per (ii) in a continuous process is not subject to any special restriction. The continuous process is preferably carried out using, for example, a fixed bed catalyst, in which case the catalyst used as per (ii) is preferably a molding as described above in Embodiments 16, 17 and 34 to 36, comprising the zeolitic material of framework structure MWW and preferably one or more than one binder material, preferably silicon dioxide. Catalyst velocity in the continuous process is preferably in the range from 0.05 to 5 mol/kg/h, more preferably from 0.1 to 4 mol/kg/h, more preferably from 0.2 to 3 mol/kg/h, catalyst velocity being defined as mol (of hydrogen peroxide)/kg (of MWW framework structure zeolitic material)/h. For a continuous process, the mixture as per (i) is preferably provided as a liquid stream which is routed into the one or more reactors and subjected therein to reaction conditions as per (ii). For the continuous process, it is also possible to route the individual components of the mixture as per (i) in the form of two or more streams, which may comprise the individual components or a mixture thereof, into the one or more reactors where the individual streams are combined after reactor entry to form the mixture as per (i). Two or more reactors operated in a continuous manner may be interconnected two or more at a time in parallel and/or two or more at a time in series. Between two reactors interconnected in series there may be provided one or more interstages, for example to intermediately recover product of value. Between two reactors interconnected in series there may further be supplied one or more of the starting materials cyclohexene, R₁OH alcohol, hydrogen peroxide and optional solvent.

Irrespective of the type of processing mode chosen, the reaction as per (ii) is performable using one or more, mutually different catalysts comprising a zeolitic material of framework structure MWVW and comprising B and Ti in the framework. The catalysts may differ, for example, with regard to the chemical composition or the manner of making the zeolitic material of framework structure MWW. When moldings are used, the catalysts may further differ for example with regard to the properties of the molding, e.g., in the geometry of the molding, the porosity of the molding, the binder content of the molding, the binder material or the percentage content of MWW framework structure zeolitic material. The reaction as per (ii) is preferably carried out in the presence of a single catalyst of the present invention.

Following the reaction as per (ii), the catalyst used is separated off from the mixture comprising the compound of formula (I). When the reaction is carried out in a continuous manner, for example in a fixed bed reactor, there is no need for a step to separate off the catalyst, since the reaction mixture leaves the reactor and the catalyst remains behind in the fixed bed reactor. When the reaction is carried out in discontinuous mode, for example in a batch reactor, the catalyst, which is preferably employed in the form of a powder, is removable using a suitable method of separation, examples being filtration, ultrafiltration, diafiltration, centrifugation and/or decanting.

Following removal, the removed catalyst may be subjected to one or more washing steps with one or more suitable washing liquids. Useful washing liquids include, for example, water, ethers such as dioxane, for example 1,4-dioxane, alcohols such as, for example, methanol, ethanol, propanol or a mixture of two or more thereof. Dioxanes are preferred for use as washing liquid. The washing step is preferably carried out at a temperature in the range from 10 to 50° C., more preferably at a temperature in the range from 15 to 40° C., more preferably at a temperature in the range from 20 to 30° C.

When the conversion rate, the selectivity or both the conversion rate and the selectivity offered by the catalyst according to the present invention decrease to below certain values, the catalyst can be regenerated in a suitable manner, for example by washing with one or more suitable washing media, or by drying in one or more suitable atmospheres, at one or more suitable temperatures and at one or more suitable pressures, or by calcination in one or more suitable atmospheres, at one or more suitable temperatures and at one or more suitable pressures, or by a combination of two or more of these measures, which may each be carried out one or more times for within one or more suitable time periods.

The reaction as per (ii) is preferably carried out at a reaction mixture temperature in the range from 40 to 150° C., more preferably at a reaction mixture temperature in the range from 50 to 125° C., more preferably at a reaction mixture temperature in the range from 70 to 100° C. When the reaction as per (ii) is carried out in a discontinuous manner, for example as a batch reaction, it is preferably carried out at the boiling point of the liquid mixture, more preferably under reflux.

When the reaction as per (ii) is carried out in a discontinuous manner, for example as a batch reaction, the duration of the reaction is preferably in the range from 1 to 12 h, more preferably in the range from 1.5 to 10 h, more preferably in the range from 2 to 8 h.

When the reaction as per (ii) is carried out in a discontinuous manner, the term “at the start of the reaction” relates generally to the point in time at which all the starting materials, including the catalyst, are simultaneously present in the reaction mixture and, depending on the temperature, the reaction of the cyclohexene starts. When the reaction as per (ii) is carried out in a continuous manner, the term “at the start of the reaction” relates generally to the point in time at which the mixture provided as per (i) comes into contact with the catalyst.

Preferably, the mole percentage for the compound of formula (I) in the mixture obtained from the reaction as per (ii), based on the sum total of mole percentages for the compounds of formulae (I), (Ib), (Ic), (Id) and (Ie)

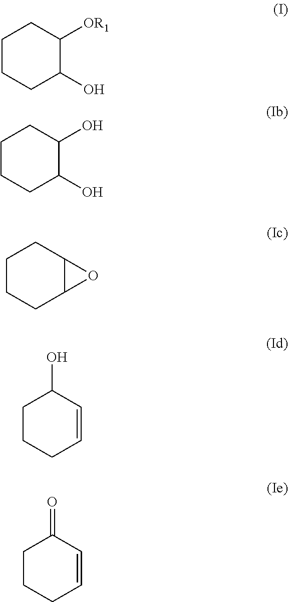

in the mixture obtained from the reaction as per (ii) is not less than 85%, preferably not less than 90%. It is not a mandatory requirement here that every one of compounds (Ib), (Ic), (Id) and (Ie) be present in the mixture obtained as per (ii); on the contrary, the mixture obtained as per (ii) may comprise just one, just two, just three or all four of the compounds (Ib), (Ic), (Id) and (Ie) in addition to the compound (I).

The mixture obtained as per (ii), comprising the compound of formula (I), is preferably worked up to recover the compound of formula (I). When the conversion of hydrogen peroxide during the reaction is incomplete, it is preferable to precede the workup by removing the unconverted hydrogen peroxide present in the mixture, for example by decomposing it through admixture of suitable substances, for example via quenching. Examples of substances suitable for decomposing the excess hydrogen peroxide include, for example, tertiary amines, polyamines, salts of heavy metals such as iron, manganese, cobalt and vanadium, sulfinic acids, mercaptans, dithionites, sulfites and strong acids and bases. The decomposition of the excess hydrogen peroxide is preferably carried out using an alkali or alkaline earth metal sulfite, more preferably alkali metal sulfite, more preferably sodium sulfite. For the purposes of the present invention it is preferably not less than 95%, more preferably not less than 97%, more preferably not less than 99%, more preferably not less than 99.5%, more preferably not less than 99.9% of the unconverted hydrogen peroxide which is removed from the mixture obtained from the reaction as per (ii).

A mixture comprising an aqueous phase and an organic phase is preferably obtained from the reaction as per (ii), preferably after removal of unconverted hydrogen peroxide. It is preferable here to precede (iii) by separating the aqueous phase from the organic phase. In principle, any batch or continuous methods known to a person skilled in the art are employable here. The organic phase separated off in this way is then employed in (iii) as the mixture obtained as per (ii).

It is possible in principle for the mixture obtained as per (ii), for example the organic phase separated off as described above, to comprise the compound of formula (Ib)

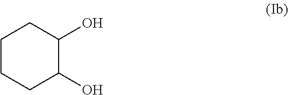

in addition to the compound of formula (I). It is conceivable in this case to precede (iii) by separating the compound of formula (Ib) in a suitable manner from the mixture obtained from the reaction as per (ii), preferably after removal of unconverted hydrogen peroxide and preferably after a step of separating off the aqueous phase.

Step (iii)

In (iii) the compound of formula (I) is suitably separated off from the mixture obtained as per (ii) to obtain a mixture concentrated in respect of the compound of formula (I). Any suitable methods of separation are employable for this step of separating off the compound of formula (Ib), although a distillative form of separation is preferable. The distillation preferably yields a mixture which is concentrated in respect of the compound of formula (I) in that it is not less than 95% by weight, preferably more than 95% by weight, for example not less than 96% by weight or not less than 97% by weight or not less than 98% by weight or not less than 99% by weight, comprised of the compound of formula (I). Optionally, this distillative step of separating off may also be used to separate off the compound of formula (Ib) and to separate off the aqueous phase in a single step.

The distillation conditions to be employed with preference are readily adaptable by a person skilled in the art to the separation problem in each case, i.e., for example to the boiling points of the compounds of formulae (I) and (Ib). Examples of preferred distillation conditions for methyl R₁ are a pot temperature in the range from 85 to 95° C. and an overhead pressure in the range from 15 to 25 mbar.

The mixture obtained by the step of separating off, concentrated with respect to the compound of formula (I), is preferably mixed with water before (iv). This aqueous mixture thus obtained is then sent into (iv) for dehydrogenation. Preference is given to aqueous mixtures comprising the compound of formula (I) at from 5% to 50% by weight, preferably at from 10% to 40% by weight, more preferably at from 15% to 35% by weight, more preferably at from 20% to 30% by weight, all based on the overall weight of the aqueous mixture.

### Step (iv)

Depending on its exact configuration, the dehydrogenation as per (iv) is in principle performable in the mixture obtained as per (iii), preferably in the aqueous mixture obtained as per (iii).

Preferably, the mixture obtained as per (iii), preferably the aqueous mixture obtained as per (iii), is suitably brought into the gas phase prior to dehydrogenation and a gas phase hydrogenation is carried out as per (iv). Preferably, therefore, the mixture which is obtained as per (iii) and which is concentrated in respect of the compound of formula (I), preferably the aqueous mixture prior to dehydrogenation as per (iv), is vaporized. Preferred temperatures for this vaporization range from 175 to 375° C., preferably from 225 to 325° C., more preferably from 250 to 300° C. Useful vaporizing apparatus includes in principle any vaporizer/evaporator known to a skilled person as suitable for this purpose.

The mixture thus preferably vaporized is then fed to the dehydrogenation as per (iv), this feeding preferably being effected using a carrier gas. Carrier gases are preferred which behave inertly or substantially inertly during the dehydrogenation as per (iv). The carrier gas is preferably selected from the group consisting of hydrogen, nitrogen, argon, carbon monoxide, water vapor and a mixture of two or more thereof, more preferably from the group consisting of hydrogen, nitrogen, argon, carbon monoxide and a mixture of two or more thereof, more preferably from the group consisting of hydrogen, nitrogen, argon and a mixture of two or more thereof. More preferable is a carrier gas comprising hydrogen and nitrogen and more preferably being not less than 95% by volume, more preferably not less than 98% by volume, more preferably not less than 99% by volume comprised of hydrogen and nitrogen. The nitrogen:hydrogen volume ratio is more preferably in the range from 2:1 to 20:1, more preferably in the range from 5:1 to 10:1.

Both the vaporizing step and the dehydrogenating step are preferably carried out in a continuous manner in the present invention.

The dehydrogenation is preferably carried out in the presence of a heterogeneous catalyst wherewith the mixture to be dehydrogenated is brought into contact. In principle, the arrangement of the catalyst in the reactor used for the dehydrogenation is not subject to any special restrictions. The heterogeneous catalyst is preferably arranged as a catalyst bed, more preferably as a fixed catalyst bed.

The chemical nature of the heterogeneous catalyst is not subject to any special restrictions beyond ensuring that the dehydrogenation of the present invention can be carried out. The dehydrogenatingly active component of the catalyst is preferably a dehydrogenatingly active noble metal, more preferably selected from the group consisting of Pd, Rh, Pt and a combination of two or more thereof.

The dehydrogenatingly active component of the catalyst, preferably the dehydrogenatingly active noble metal, is preferably supported in a suitable manner on a carrier. The carrier material is not subject to any special restrictions beyond ensuring that the carrier material behavior during the dehydrogenation is substantially inert or promotive of the dehydrogenation. The carrier material is preferably selected from the group consisting of activated carbon, aluminum oxide, silicon oxide and a combination of two or more thereof.

One preferred catalyst for the purposes of the present invention comprises Pd as dehydrogenatingly active component and activated carbon as carrier material, the palladium content of the catalyst preferably being in the range from 1% to 10% by weight, more preferably in the range from 2% to 8% by weight, more preferably in the range from 3% to 7% by weight, all based on the overall mass of the catalyst.

In another catalyst preferred for the purposes of the present invention and comprising Pd as dehydrogenatingly active component and activated carbon as carrier material, the palladium content of the catalyst is preferably in the range from 0.1% to 5% by weight, more preferably in the range from 0.2% to 3% by weight, more preferably in the range from 0.5% to 2% by weight, all based on the overall mass of the catalyst.

The heterogeneous catalyst is preferably activated in a suitable manner prior to dehydrogenation as per (iv). As part of this activation, the catalyst is preferably flushed with a gas, preferably at an elevated temperature as compared with room temperature. The gas employed for flushing is preferably selected from the group consisting of hydrogen, nitrogen, argon and a mixture of two or more thereof, more preferably from the group consisting of hydrogen, nitrogen and a mixture thereof. It is inter alia preferable to flush first with a mixture of hydrogen and nitrogen wherein the volume ratio of nitrogen:hydrogen is preferably in the range from 10:1 to 30:1, more preferably in the range from 15:1 to 25:1, and then with hydrogen. Preferred temperatures for flushing are in the range from 300 to 550° C., more preferably from 350 to 500° C., more preferably from 375 to 450° C. These temperatures are the temperature of the gas, or gas mixture, used for flushing the catalyst. The catalyst may in principle be flushed outside the reactor used for dehydrogenation. The catalyst is preferably flushed inside the reactor used for dehydrogenation.

Before or after this flushing, preferably before this flushing, the catalyst is washable in a suitable manner, preferably with an aqueous solution comprising a base, preferably a hydroxide, more preferably an alkali metal hydroxide, more preferably potassium hydroxide. An example of what is preferable is a wash with an aqueous solution of an alkali metal hydroxide, preferably potassium hydroxide, having an alkali metal hydroxide content in the range from 0.2% to 7% by weight, preferably from 0.5% to 6% by weight, more preferably from 1% to 5% by weight, based on the overall weight of the aqueous solution.

Prior to the actual hydrogenation, the reactor used for the dehydrogenation and comprising the catalyst is preferably suitably purged with a gas which is preferably selected from the group consisting of nitrogen, argon and a mixture thereof, more preferably comprises nitrogen, more preferably is technical-grade nitrogen.

The temperature at which the dehydrogenation as per (iv) is carried out is preferably in the range from 200 to 400° C., more preferably from 250 to 350° C., more preferably from 275 to 325° C. This temperature is to be understood as the temperature of the catalyst employed for the dehydrogenation. When the catalyst is, as is preferable, in the form of a catalyst bed, more preferably in the form of a fixed catalyst bed, this temperature is to be understood as meaning the temperature of the fixed catalyst bed.

Preferably, the formylating step as per (v) is preceded by the compound of formula (II) being separated off from the mixture obtained after dehydrogenation as per (iv) to obtain a mixture concentrated in respect of the compound of formula (II). Any suitable methods of separation are employable for this step of separating off the compound of formula (II), although a distillative form of separation is preferable. The distillation preferably yields a mixture which is concentrated in respect of the compound of formula (II) in that it is not less than 95% by weight, preferably more than 95% by weight, for example not less than 96% by weight or not less than 97% by weight or not less than 98% by weight or not less than 99% by weight, comprised of the compound of formula (II).

The distillation conditions to be employed with preference are readily adaptable by a person skilled in the art to the separation problem in each case. Examples of preferred distillation conditions for methyl R₁ are a pot temperature in the range from 55 to 80° C. and an overhead pressure in the range from 0.5 to 5 mbar. It is preferably the mixture concentrated as per the compound of formula (II) which is separated off at the top of the column.

### Step (v)

Step (v) comprises formylating the formula (II) compound present in the mixture obtained as per (iv) to obtain a mixture comprising the compound of formula (IV).

This formylation may in general be carried out according to any suitable procedure. The invention preferably comprises reacting the formula (II) compound present in the mixture obtained as per (iv) with glyoxylic acid, to obtain a mixture comprising the compound of formula (III)

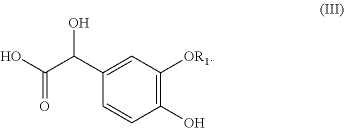

The preferred starting material for this is an aqueous solution comprising the glyoxylic acid. Preference is given to aqueous solutions whose glyoxylic acid content is in the range from 30% to 70% by weight, preferably from 40% to 60% by weight. The reaction with glyoxylic acid is accordingly with preference carried out in aqueous phase. The present invention accordingly provides the method as described above and wherein the formylating step as per (v) comprises:
(v-1) reacting the formula (II) compound present in the mixture obtained as per (iv) with glyoxylic acid OHC—COOH, preferably in aqueous phase, to obtain a mixture comprising a compound of formula (III)

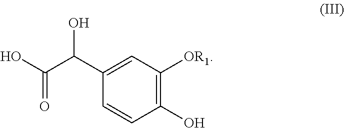

The reaction as per (v-1) more preferably takes place in a basic medium. The preference for this is for the reaction mixture to comprise a Bronstedt base, preferably selected from the group consisting of alkali metal hydroxides, alkaline earth metal hydroxides and a mixture of two or more thereof, more preferably from the group consisting of alkali metal hydroxides and a mixture of two or more thereof. The reaction mixture more preferably comprises sodium hydroxide.

In a preferred embodiment of the method according to the present invention, an initial charge comprises an aqueous solution comprising glyoxylic acid and preferably further comprising a Bronstedt base, preferably selected from the group consisting of alkali metal hydroxides, alkaline earth metal hydroxides and a mixture of two or more thereof, more preferably from the group consisting of alkali metal hydroxides and a mixture of two or more thereof, wherein the Bronstedt base more preferably comprises sodium hydroxide and more preferably is sodium hydroxide. This aqueous solution is then preferably mixed with an aqueous solution comprising the mixture obtained as per (iv) and preferably a Bronstedt base, preferably selected from the group consisting of alkali metal hydroxides, alkaline earth metal hydroxides and a mixture of two or more thereof, more preferably from the group consisting of alkali metal hydroxides and a mixture of two or more thereof, wherein the Bronstedt base more preferably comprises sodium hydroxide and more preferably is sodium hydroxide. More preferably, in the resulting mixture, the molar ratio of Bronstedt base, more preferably sodium hydroxide, to the sum total formed from the compound of formula (II) and from glyoxylic acid is in the range from 0.2:1 to 2:1, preferably from 0.5:1 to 1.5:1, more preferably from 0.75:1 to 1.25:1.

The reaction as per (v-1) is preferably carried out at a reaction mixture temperature in the range from 10 to 40° C., more preferably in the range from 15 to 35° C., more preferably in the range from 20 to 30° C. Suitable stirring is more preferably applied to the reaction mixture during said reaction. The pH of the mixture obtained after the reaction as per (v-1) is preferably in the range from 9.5 to 12.5, more preferably from 10 to 12, more preferably from 10.5 to 11.5.

The mixture obtained as per (v-1) is preferably purified in a further step to obtain a mixture that is concentrated in respect of the compound of formula (III). This purification is preferably carried out such that the mixture obtained, purified in respect of the compound of formula (III), is an aqueous mixture. The present invention accordingly provides the method as described above and wherein the formylating step as per (v) further comprises:
(v-2) purifying the mixture obtained as per (v-1) in respect of the compound of formula (III) to obtain a preferably aqueous mixture comprising the compound of formula (III).

In principle, this purification as per (v-2) is not subject to any special restriction. Preferably, this purification comprises an extraction with one or more suitable organic solvents, more preferably comprising toluene. It is further preferable for the purposes of the present invention to use toluene to extract the mixture obtained as per (v-1). Preferably unconverted compound of formula (II) is extracted from the aqueous phase in the course of this extraction. The formula (II) compound thus separated off is then advantageously recyclable into the method of the present invention for use as starting material for the formylation as per (v). The present invention accordingly provides the method as described above and wherein the purification as per (v-2) comprises an extraction, preferably an extraction with an organic solvent, preferably comprising toluene.

It is further preferable for the purposes of the present invention for the mixture obtained as per (v-1) to be suitably acidified before said extraction and to perform said extraction on the basis of a mixture having a lower pH. Preferably, the pH of the mixture is adjusted to a value in the range from 0.5 to 1.5 in the course of (v-2). More preferably, the pH adjustment to a value in the range from 0.5 to 1.5 is effected in two or more steps, preferably in two steps. The extraction referred to is preferably carried out after the first step. The pH of the mixture after this first step is preferably in the range from 4.0 to 5.0.

Any suitable acids are in principle usable for adjusting the pH. The use of sulfuric acid is particularly preferable. pH here is to be understood as meaning the pH as determined on using a pH-sensitive glass electrode.

What the present invention accordingly provides as per (v-2) is preferably an aqueous mixture preferably having a pH in the range from 0.5 to 1.5 and comprising the compound of formula (III).

The formula (III) compound present in the mixture obtained as per (v-1), preferably as per (v-2), is preferably, in a further step of the present invention, subjected to an oxidative decarboxylation to obtain a mixture comprising the compound of formula (IV). The present invention accordingly provides the method as described above and wherein the formylating step as per (v) further comprises:
(v-3) oxidatively decarboxylating the formula (III) compound present in the mixture obtained as per (v-1), preferably as per (v-2), to obtain a mixture comprising the compound of formula (IV).

The oxidative decarboxylation is preferably preceded by the aqueous mixture used, obtained as per (v-1), preferably as per (v-2), being mixed with one or more organic solvents, preferably comprising toluene, so the mixture used in (v-3), which comprises the compound of formula (III), is an aqueous-organic mixture.

The step of oxidatively decarboxylating as per (v-3) is preferably carried out in the presence of an oxidizing agent. Any suitable oxidizing agents are usable in principle. The oxidizing agent is preferably selected from the group consisting of CuO, PbO₂, MnO₂, Co₃O₄, HgO, Ag₂O, Cu(II) salts, Hg(II) salts, Fe(III) salts, Ni(III) salts, Co(III) salts, chlorates and a mixture of two or more thereof, preferably from the group consisting of CuO, MnO₂, Cu(II) salts, Fe(III) salts and a mixture of two or more thereof. The oxidative decarboxylation as per (v-3) is more preferably carried out in the presence of FeCl₃ as oxidizing agent.

Preferred temperatures at which the oxidative decarboxylation as per (v-3) is carried out range from 60 to 110° C., more preferably from 70 to 100° C., more preferably from 80 to 95° C. This temperature is to be understood as meaning the temperature of the reaction mixture.

Step (v), as described above, provides a mixture, preferably an aqueous mixture, more preferably an aqueous-organic mixture comprising the compound of formula (IV). This mixture is in principle usable as such for further reactions when the formula (IV) compound present in the mixture, for example vanillin, ethylvanillin or isopropylvanillin, serves as intermediate for subsequent steps of synthesis and the aqueous or aqueous-organic mixture is suitable therefor. The present invention accordingly also provides a mixture comprising the compound of formula (IV), obtained or obtainable as per any one above-described method comprising (i) to (v), preferably comprising (i) to (v-3).

### Step (vi)

It is preferable for the purposes of the present invention for the compound of formula (IV) to be separated off in a suitable manner from the mixture obtained as per (v), preferably (v-3), and preferably be purified thereby. The present invention accordingly also provides the above-described method further comprising
(vi) separating the compound of formula (IV) off from the mixture obtained as per (v), preferably from the mixture obtained as per (v-3).

In principle, any procedures known to a person skilled in the art are employable for this step of separating off. It is particularly preferable to take the aqueous-organic phase more preferably obtained as per (v-3) and separate off the organic phase, comprising the compound of formula (IV), in a suitable manner. The present invention accordingly provides the above-described method wherein the mixture obtained as per (v), preferably as per (v-3) comprises water and one or more than one organic solvent, and wherein (vi) comprises:
(vi-1) separating the organic phase from the aqueous phase to obtain an organic phase comprising the compound of formula (IV).

Any procedure known to a person skilled in the art is employable to separate off the organic phase comprising the compound of formula (IV), preferably further comprising an organic solvent, preferably toluene.

The aqueous phase separated off from the organic phase as per the preferred method recited is preferably extracted with an organic solvent, preferably comprising toluene, to transfer into an organic phase any formula (IV) compound still present in the aqueous phase. Preference here is for the extracting step to be carried out at an elevated temperature as compared with room temperature for the mixture to be extracted, the temperature more preferably being in the range from 50 to 95° C., more preferably from 60 to 95° C., more preferably from 70 to 95° C., more preferably from 80 to 95° C., more preferably from 85 to 95° C. The one or more than one organic solvent used preferably comprises toluene and more preferably is toluene. The present invention accordingly also provides the above-described method wherein (vi) further comprises:
(vi-2) extracting the aqueous phase with one or more than one organic solvent to obtain one or more than one further organic phase comprising the compound of formula (IV).

It is generally conceivable for this removed organic phase as per (vi-1) or the removed organic phase as per (vi-1) or the combined organic phases as per (vi-1) and (vi-2), comprising the compound of formula (IV), to be used as such, for instance when the compound of formula (IV) serves as intermediate for subsequent steps of synthesis. It is preferable for the purposes of the present invention for the organic phase as per (vi-1), or the combined organic phases as per (vi-1) and (vi-2), to be washed in a further step wherein the washing medium preferably comprises water, more preferably is water. This washing step is preferably carried out at a washing medium temperature in the range from 10 to 40° C., preferably from 15 to 35° C., more preferably from 20 to 30° C. The present invention accordingly also provides the above-described method wherein (vi) further comprises:
(vi-3) washing the organic phase or phases comprising the compound of formula (IV) and obtained as per (vi-1) or, respectively, as per (vi-1) and (vi-2).

It is generally conceivable for the preferably washed organic phase comprising the compound of the formula (IV) to be used as such, for instance when the compound of formula (IV) serves as intermediate for subsequent steps of synthesis. It is preferred for the purposes of the present invention for the organic phase obtained as per (vi-1), or the organic phases obtained as per (vi-1) and (vi-2), preferably the organic phase washed as per (vi-3), or the organic phases washed as per (vi-3), to be concentrated in respect of the compound of formula (IV) to further preferably obtain the compound of formula (IV). The term “the compound of formula (IV)” as used in this context signifies a mixture or composition preferably not less than 99% by weight, based on the overall weight of the mixture or composition, comprised of the compound of formula (IV). And the procedure used for this concentrating step is not subject to any special restrictions. A concentrating step is preferably carried out under a pressure reduced from ambient pressure to preferably within the range from 1 to 100 mbar, more preferably from 1 to 50 mbar, more preferably from 1 to 10 mbar. The present invention accordingly also provides the above-described method wherein (vi) further comprises:
(vi-4) concentrating the organic phase obtained as per (vi-1) or the organic phases obtained as per (vi-1) and (vi-2), preferably the organic phase washed as per (vi-3) or the organic phases washed as per (vi-3), in respect of the compound of formula (IV).

The present invention is further illustrated by the following embodiments and combinations of embodiments apparent from dependencies and other references:

1. A method of preparing a compound of formula (IV)

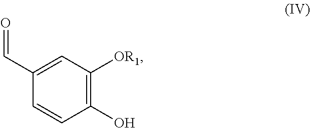

where R₁ is alkyl of 1 to 4 carbon atoms, comprising
(i) providing a liquid mixture comprising cyclohexene, an alcohol R₁OH, hydrogen peroxide and optionally a solvent;
(ii) reacting the cyclohexene with the hydrogen peroxide and the alcohol R₁OH in the mixture provided as per (i) in the presence of a catalyst comprising a zeolite of framework structure MWW to obtain a mixture comprising the compound of formula (I)

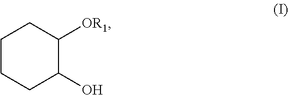

where the framework of the zeolite as per (ii) comprises silicon, titanium, boron, oxygen and hydrogen;
(iii) separating the compound of formula (I) off from the mixture obtained as per (ii) to obtain a mixture concentrated in respect of the compound of formula (I);
(iv) dehydrogenating the formula (I) compound present in the concentrated mixture obtained as per (iii) to obtain a mixture comprising a compound of formula (II)

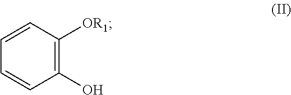

(v) formylating the formula (II) compound present in the mixture obtained as per (iv) to obtain a mixture comprising the compound of formula (IV).
2. The method according to Embodiment 1 wherein R₁ is methyl or ethyl or isopropyl, preferably methyl or ethyl.
3. The method according to Embodiment 1 or 2 wherein R₁ is methyl.
4. The method according to any one of Embodiments 1 to 3 wherein the liquid mixture provided as per (i) has a molar ratio prior to the reaction as per (ii) of cyclohexene:R₁OH in the range from 1:1 to 1:50, preferably from 1:3 to 1:30.
5. The method according to Embodiment 4 wherein the liquid mixture provided as per (i) has a molar ratio prior to the reaction as per (ii) of cyclohexene:R₁OH in the range from 1:5 to 1:10.
6. The method according to any one of Embodiments 1 to 5 wherein the liquid mixture provided as per (i) has a molar ratio prior to the reaction as per (ii) of cyclohexene:hydrogen peroxide in the range from 1:1 to 5:1, preferably from 1.5:1 to 4.5:1.
7. The method according to Embodiment 6 wherein the liquid mixture provided as per (i) has a molar ratio prior to the reaction as per (ii) of cyclohexene:hydrogen peroxide in the range from 2:1 to 4:1.
8. The method according to any one of Embodiments 1 to 7 wherein the liquid mixture provided as per (i) comprises a solvent.
9. The method according to Embodiment 8 wherein the solvent is selected from the group consisting of C1-C6-alkyl nitriles, dialkyl ketones of the formula R₂—CO—R₃, where R₂ and R₃ are each independently selected from the group consisting of C1-C6-alkyl, and a mixture of two or more thereof, more preferably from the group consisting of C1-C3-alkyl nitriles, dialkyl ketones of the formula R₂—CO—R₃, where R₂ and R₃ are each independently selected from the group consisting of C1-C3-alkyl, and a mixture of two or more thereof.
10. The method according to Embodiment 8 or 9 wherein the solvent is selected from the group consisting of acetonitrile, acetone and a mixture thereof.
11. The method according to any one of Embodiments 8 to 10 wherein the molar ratio of solvent:cyclohexene in the liquid mixture provided as per (i) is in the range from 20:1 to 1:1, preferably from 15:1 to 1:1, before the reaction as per (ii) subject to the proviso that when the solvent in the mixture comprises a mixture of two or more solvents, the molar ratio of solvent:cyclohexene is based on the mixture of solvents.
12. The method according to Embodiment 11 wherein the molar ratio of solvent:cyclohexene in the liquid mixture provided as per (i) is in the range from 10:1 to 1:1, before the reaction as per (ii) subject to the proviso that when the solvent in the mixture comprises a mixture of two or more solvents, the molar ratio of solvent:cyclohexene is based on the mixture of solvents.
13. The method according to any one of Embodiments 1 to 7 wherein the liquid mixture provided as per (i) comprises no solvent.
14. The method according to any one of Embodiments 1 to 13 wherein the liquid mixture as per (i) is provided by admixing hydrogen peroxide to a mixture comprising the cyclohexene, the alcohol R₁OH and optionally the solvent.
15. The method according to Embodiment 14 wherein the hydrogen peroxide is admixed in the form of an aqueous solution, wherein the hydrogen peroxide content of the aqueous solution is preferably in the range from 25 to 75 wt %, more preferably from 40 to 70 wt %, based on the overall weight of the aqueous solution.
16. The method according to any one of Embodiments 1 to 15 wherein the reaction as per (ii) is carried out at a temperature in the range from 40 to 150° C., preferably from 50 to 125° C.
17. The method according to Embodiment 16 wherein the reaction as per (ii) is carried out at a temperature in the range from 70 to 100° C.
18. The method according to any one of Embodiments 1 to 17 wherein the reaction as per (ii) is carried out at the boiling point of the liquid mixture, preferably under reflux.
19. The method according to any one of Embodiments 1 to 18 wherein the duration of the reaction as per (ii) is in the range from 1 to 12 h, preferably from 1.5 to 10 h.
20. The method according to Embodiment 19 wherein the duration of the reaction as per (ii) is in the range from 2 to 8 h.
21. The method according to any one of Embodiments 1 to 20 wherein the mass ratio of hydrogen peroxide:zeolite of framework structure MWW at the start of the reaction as per (ii) is in the range from 10:1 to 0.1:1, preferably from 1:1 to 0.2:1.
22. The method according to Embodiment 21 wherein the mass ratio of hydrogen peroxide:zeolite of framework structure MWW at the start of the reaction as per (ii) is in the range from 0.75:1 to 0.25:1.
23. The method according to any one of Embodiments 1 to 22 wherein the reaction as per (ii) is carried out as a batch process.
24. The method according to any one of Embodiments 1 to 22 wherein the reaction as per (ii) is carried out as a continuous process.
25. The method according to any one of Embodiments 1 to 24 wherein not less than 99 wt %, preferably not less than 99.5 wt % of the framework of the zeolite as per (ii) consists of silicon, titanium, boron, oxygen and hydrogen.
26. The method according to Embodiment 25 wherein not less than 99.9 wt % of the framework of the zeolite as per (ii) consists of silicon, titanium, boron, oxygen and hydrogen.
27. The method according to any one of Embodiments 1 to 26 wherein, in the zeolite of framework structure MWW, the molar ratio of B:Si is in the range from 0.02:1 to 0.5:1, preferably from 0.05:1 to 0.15:1.
28. The method according to any one of Embodiments 1 to 27 wherein, in the zeolite of framework structure MWW, the molar ratio of Ti:Si is in the range from 0.01:1 to 0.05:1, preferably from 0.017:1 to 0.025:1.
29. The method according to any one of Embodiments 1 to 28 wherein the zeolite of framework structure MWW is obtainable or obtained as per a method comprising
   (a) providing an aqueous synthesis mixture comprising a silicon source, a boron source, a titanium source and an MWW-templating compound, wherein the temperature of the aqueous synthesis mixture is not more than 50° C.;
   (b) heating the aqueous synthesis mixture provided as per (a) from the temperature of not more than 50° C. to a temperature in the range from 160 to 190° C. in the course of a period of at most 24 h;
   (c) subjecting the synthesis mixture as per (b) to hydrothermal synthesis conditions under autogenous pressure in a closed system at a temperature in the range from 160 to 190° C. to obtain a precursor to the zeolite of framework structure MWW in its mother liquor;
   (d) separating the precursor to the zeolite of framework structure MWW off from its mother liquor;
   (e) calcining the MWWV framework structure zeolite precursor separated off as per (d) to obtain the zeolite of framework structure MWW.
30. The method according to any one of Embodiments 1 to 29 wherein the catalyst, preferably in the form of a molding, comprises a binder, preferably a silica binder, in addition to the MWW framework structure zeolite.
31. The method according to any one of Embodiments 1 to 30 wherein the mixture provided as per (i) comprises no sulfuric acid, preferably no strong nonnucleophilic acid.
32. The method according to any one of Embodiments 1 to 30 wherein the mole percentage for the compound of formula (I) in the mixture obtained from the reaction as per (ii), based on the sum total of mole percentages for the compounds of formulae (I), (Ib), (Ic), (Id) and (Ie)

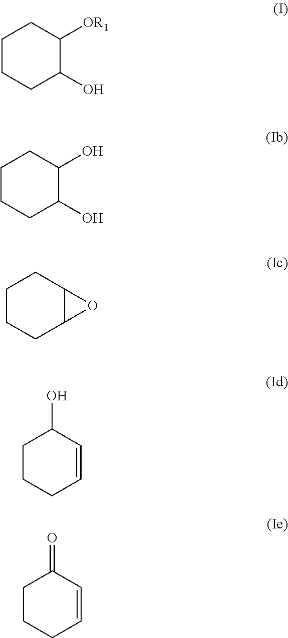

in the mixture obtained from the reaction as per (ii) is not less than 85%, preferably not less than 90%.
33. The method according to any one of Embodiments 1 to 32 wherein the mixture obtained from the reaction as per (ii) comprises unconverted hydrogen peroxide, and wherein (ii) further comprises removing at least some, preferably not less than 95% of the unconverted hydrogen peroxide from the mixture obtained from the reaction as per (ii)
34. The method according to Embodiment 33 wherein the mixture obtained from the reaction as per (ii) comprises unconverted hydrogen peroxide, and wherein (ii) further comprises removing not less than 99% of the unconverted hydrogen peroxide from the mixture obtained from the reaction as per (ii).
35. The method according to Embodiment 33 or 34 wherein the step of removing the unconverted hydrogen peroxide comprises quenching the unconverted hydrogen peroxide.
36. The method according to Embodiment 35 wherein the step of removing the unconverted hydrogen peroxide comprises quenching the unconverted hydrogen peroxide with sodium sulfite.
37. The method according to any one of Embodiments 1 to 36 wherein the mixture obtained from the reaction as per (ii), preferably the mixture obtained after the step of removing the unconverted hydrogen peroxide, comprises an aqueous phase and an organic phase, and wherein said method further comprises the step of separating off the organic phase from the aqueous phase, wherein the organic phase separated off is employed in (iii) as mixture obtained as per (ii).
38. The method according to any one of Embodiments 1 to 37 wherein the compound of formula (Ib)

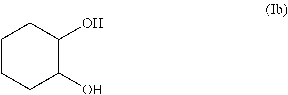

is comprised in the mixture obtained from the reaction as per (ii),
39. The method according to any one of Embodiments 1 to 38 wherein as per (iii) the step of separating the compound of formula (I) off from the mixture obtained as per (ii) comprises a distillation wherein the mixture obtained from the distillation and concentrated in respect of the compound of formula (I) comprises the compound of formula (I) at preferably not less than 90 wt %, more preferably at not less than 95 wt %.
40. The method according to Embodiment 39 wherein as per (iii) the step of separating the compound of formula (I) off from the mixture obtained as per (ii) comprises a distillation wherein the mixture obtained from the distillation and concentrated in respect of the compound of formula (I) comprises the compound of formula (I) at more than 95 wt %.
41, The method according to any one of Embodiments 1 to 40 wherein the mixture obtained as per (iii), which is concentrated in respect of the compound of formula (I), is mixed with water, to obtain an aqueous mixture, before the dehydrogenation as per (iv).
42. The method according to Embodiment 41 wherein the aqueous mixture obtained comprises the compound of formula (I) at from 5% to 50% by weight, preferably at from 10% to 40% by weight, based on the overall weight of the aqueous mixture.
43. The method according to Embodiment 42 wherein the aqueous mixture obtained comprises the compound of formula (I) at from 20% to 30% by weight, based on the overall weight of the aqueous mixture.
44. The method according to any one of Embodiments 1 to 43, preferably according to any one of Embodiments 41 to 43, wherein the mixture obtained as per (iii), concentrated in respect of the compound of formula (I), preferably the aqueous mixture as per any one of Embodiments 41 to 43, is vaporized, preferably at a temperature in the range from 175 to 375° C., more preferably from 225 to 325° C., prior to the dehydrogenation as per (iv).
45. The method according to Embodiment 44 wherein the mixture obtained as per (iii), concentrated in respect of the compound of formula (I), preferably the aqueous mixture as per any one of Embodiments 41 to 43, is vaporized, at a temperature in the range from 250 to 300° C., prior to the dehydrogenation as per (iv).
46. The method according to Embodiment 44 or 45 wherein the vaporized mixture is fed to the dehydrogenating step using a carrier gas, wherein the carrier gas is preferably selected from the group consisting of hydrogen, nitrogen, argon, carbon monoxide, water vapor and a mixture of two or more thereof.
47. The method according to Embodiment 46 wherein the carrier gas comprises a mixture of hydrogen and nitrogen, preferably is not less than 95% by volume, more preferably not less than 98% by volume comprised of hydrogen and nitrogen.
48. The method according to any one of Embodiments 1 to 47 wherein the dehydrogenation as per (iv) is carried out in the presence of a heterogeneous catalyst, wherein the catalyst preferably comprises a dehydrogenatingly active noble metal, more preferably a noble metal selected from the group consisting of Pd, Rh, Pt and a combination of two or more thereof.
49. The method according to Embodiment 48 wherein the noble metal is preferably supported by one or more than one carrier material, which is preferably selected from the group consisting of activated carbon, aluminum oxide, silicon oxide and a combination of two or more thereof.
50. The method according to Embodiment 48 or 49 wherein the catalyst comprises palladium supported on activated carbon.
51. The method according to any one of Embodiments 48 to 50 wherein the heterogeneous catalyst is activated before the dehydrogenation as per (iv).
52. The method according to Embodiment 51 wherein the step of activating comprises the step of flushing the heterogeneous catalyst with a gas preferably selected from the group consisting of hydrogen, nitrogen, argon and a mixture of two or more thereof.
53. The method according to Embodiment 52 wherein the step of flushing with the gas is preferably carried out at a gas temperature in the range from 350 to 500° C., more preferably from 375 to 450° C.
54. The method according to Embodiment 52 or 53 wherein the step of activating comprises the step of washing the catalyst with an aqueous solution comprising a base, preferably a hydroxide, more preferably an alkali metal hydroxide, more preferably potassium hydroxide.
55. The method according to any one of Embodiments 48 to 54 wherein the dehydrogenation as per (iv) is carried out at a catalyst temperature in the range from 200 to 400° C., preferably from 250 to 350° C., more preferably from 275 to 325° C.
56. The method according to Embodiment 55 wherein the catalyst is used in the form of a catalyst bed and it is the temperature of the catalyst bed which is the catalyst temperature.
57. The method according to any one of Embodiments 1 to 56 wherein the formylating step as per (v) is preceded by the compound of formula (II) being separated off from the mixture obtained from (iv) to obtain a mixture concentrated in respect of the compound of formula (II).
58. The method according to Embodiment 57 wherein the step of separating off comprises a distillation and preferably is a distillation.
59. The method according to Embodiment 57 or 58 wherein the mixture obtained, which is concentrated in respect of the compound of the formula (II), comprises the compound of formula (II) at not less than 95 wt %, more preferably at not less than 98 wt %.
60. The method according to any one of Embodiments 1 to 59 wherein the formylating step as per (v) comprises:
   (v-1) reacting the formula (II) compound present in the mixture obtained as per (iv) with glyoxylic acid OHC—COOH, preferably in aqueous phase, to obtain a mixture comprising a compound of formula (III)

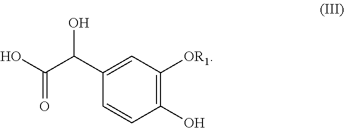

61. The method according to Embodiment 60 wherein the step of reacting as per (v-1) is carried out in the presence of a Bronstedt base, preferably selected from the group consisting of alkali metal hydroxides, alkaline earth metal hydroxides and a mixture of two or more thereof, more preferably from the group consisting of alkali metal hydroxides and a mixture of two or more thereof.
62. The method according to Embodiment 61 wherein the Bronstedt base comprises sodium hydroxide and preferably is sodium hydroxide.
63. The method according to any one of Embodiments 60 to 62 wherein the step of reacting as per (v-1) is carried out at a temperature in the range from 10 to 40° C., preferably in the range from 15 to 35° C.
64. The method according to Embodiment 63 wherein the step of reacting as per (v-1) is carried out at a temperature in the range from 20 to 30° C.
65. The method according to any one of Embodiments 60 to 64 wherein the formylating step as per (v) comprises further:
   (v-2) purifying the mixture obtained as per (v-1) in respect of the compound of formula (III) to obtain a preferably aqueous mixture comprising the compound of formula (III).
66. The method according to Embodiment 65 wherein the step of purifying comprises an extraction.
67. The method according to Embodiment 65 or 66 wherein the pH of the mixture obtained from the reaction as per (v-1) is adjusted during (v-2) to a value in the range from 0.5 to 1.5.
68. The method according to Embodiment 67 wherein the pH is adjusted in two steps, wherein a first step comprises adjusting the pH of the mixture obtained from the reaction as per (v-1) to a value in the range from 4.0 to 5.0 and a second step comprises adjusting the pH to a value in the range from 0.5 to 1.5, wherein the formula (II) compound present in the mixture obtained as per (ii) is separated off from the mixture, having a pH in the range from 4.0 to 5.0, after the first step.
69. The method according to Embodiment 68 wherein the formula (II) compound present in the mixture obtained as per (ii) is separated off by extraction from the mixture, having a pH in the range from 4.0 to 5.0, after the first step.
70. The method according to any one of Embodiments 60 to 69 wherein the formylating step as per (v) comprises further:
   (v-3) oxidatively decarboxylating the formula (III) compound present in the mixture obtained as per (v-1), preferably as per (v-2), to obtain a mixture comprising the compound of formula (IV).
71. The method according to claim 70 wherein the step of oxidatively decarboxylating as per
   (v-3) is carried out in the presence of an oxidizing agent selected from the group consisting of CuO, PbO₂, MnO₂, Co₃O₄, HgO, Ag₂O, Cu(II) salts, Hg(II) salts, Fe(III) salts, Ni(III) salts, Co(III) salts, chlorates and a mixture of two or more thereof, preferably from the group consisting of CuO, MnO₂, Cu(II) salts, Fe(III) salts and a mixture of two or more thereof.
72. The method according to Embodiment 71 wherein the oxidative decarboxylation as per (v-3) is carried out in the presence of FeCl₃ as oxidizing agent.
73. The method according to any one of Embodiments 70 to 72 wherein the step of oxidatively decarboxylating as per (v-3) is carried out at a temperature in the range from 60 to 110° C., preferably from 70 to 100° C.
74. The method according to Embodiment 73 wherein the step of oxidatively decarboxylating as per (v-3) is carried out at a temperature in the range from 80 to 95° C.
75. The method according to any one of Embodiments 1 to 74 comprising
   (vi) separating the compound of formula (IV) off from the mixture obtained as per (v), preferably from the mixture obtained as per (v-3).
76. The method according to Embodiment 75 wherein the mixture obtained as per (v), preferably as per (v-3) comprises water and one or more than one organic solvent, and wherein (vi) comprises:
   (vi-1) separating the organic phase from the aqueous phase to obtain an organic phase comprising the compound of formula (IV).
77. The method according to Embodiment 76 wherein the one or more than one organic solvent comprises toluene and preferably is toluene.
78. The method according to Embodiment 76 or 77 wherein (vi) further comprises:
   (vi-2) extracting the aqueous phase with one or more than one organic solvent to obtain one or more than one further organic phase comprising the compound of formula (IV).
79. The method according to Embodiment 78 wherein the one or more than one organic solvent comprises toluene and preferably is toluene.
80. The method according to any one of Embodiments 76 to 79 wherein (vi) further comprises:
   (vi-3) washing the organic phase or phases comprising the compound of formula (IV) and obtained as per (vi-1) or, respectively, as per (vi-1) and (vi-2).
81, The method according to Embodiment 80 wherein the step of washing is preferably carried out with at least one washing medium that preferably comprises water and more preferably is water.
82. The method according to any one of Embodiments 76 to 81 wherein (vi) further comprises:
   (vi-4) concentrating the organic phase obtained as per (vi-1) or the organic phases obtained as per (vi-1) and (vi-2), preferably the organic phase washed as per (vi-3) or the organic phases washed as per (vi-3), in respect of the compound of formula (IV).
83. The method according to Embodiment 82 wherein the step of concentrating as per (iv-4) is carried out under reduced pressure as compared with ambient pressure.
84. A mixture comprising the compound of formula (IV)

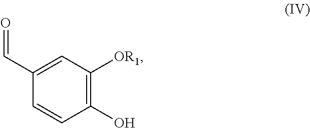

where R₁ is alkyl of 1 to 4 carbon atoms, obtained or obtainable by a method according to any one of Embodiments 1 to 83, preferably according to any one of Embodiments 75 to 83, more preferably according to Embodiment 82 or 83.

The invention is more particularly elucidated in the examples and reference examples which follow.

### EXAMPLES

The experimental examples which follow utilized the following starting materials:

1. piperidine (Sigma-Aldrich)

2. boric acid (Bernd Kraft)

3. tetrabutyl orthotitanate (Alfa Aesar)

4. CAB-O-SIL® M7D and CAB-O-SIL® M5 (Cabot) fumed silica

5. Ludox® AS-40 (Sigma-Aldrich) colloidal silica

6. cyclohexene (Sigma Aldrich)

7. methanol (Fluka)

8. hydrogen peroxide (Solvay)

### Reference Example 1: Measurement of ¹¹B Solid State NMR Spectra

The ¹¹B solid state NMR experiments were carried out using a Bruker Avance III spectrometer operating at 400 MHz ¹H Larmor frequency (Bruker Biospin, Germany). The samples were stored at room temperature and 63% relative humidity before being packed into 4 mm ZrO₂ rotors. Measurements were performed under 10 kHz magic angle spinning at room temperature. ¹¹B-Spectra were obtained using ¹¹B 15° pulse excitation of 1 microsecond (μs) pulse width, an ¹¹B carrier frequency corresponding to −4 ppm in the referenced spectrum, and a scan recycle delay of 1 s. Signal was acquired for 10 ms and accumulated with 5000 scans. Spectra were processed using a Bruker Topspin with 30 Hz exponential line broadening, phasing and baseline correction across the full width of the spectrum. Spectra were indirectly referenced to 1% TMS in CDCl₃ on the unified chemical shift scale according to IUPAC (Pure Appl. Chem., vol. 80, No. 1, p. 59) using glycine with carbonyl peak at 175.67 ppm as secondary standard.

### Reference Example 2: Measurement of ²⁹Si Solid State NMR Spectra

²⁹Si solid state NMR experiments were carried out using a Bruker Advance III spectrometer operating at 400 MHz ¹H Larmor frequency (Bruker Biospin, Germany). The samples were stored at room temperature and 63% relative humidity before being packed into 4 mm ZrO₂ rotors. Measurements were performed under 10 kHz magic angle spinning at room temperature. ²⁹Si-Spectra were obtained using ²⁹Si 90° pulse excitation of 5 microsecond (μs) pulse width, a ²⁹Si carrier frequency corresponding to −112 ppm in the referenced spectrum, and a scan recycle delay of 120 s. Signal was acquired for 20 milliseconds (ms) at 63 kHz high-power proton decoupling and accumulated for at least 16 hours. Spectra were processed using a Bruker Topspin with 50 Hz exponential line broadening, phasing and baseline correction across the full width of the spectrum. Spectra were indirectly referenced to 1% TMS in CDCl₃ on the unified chemical shift scale according to IUPAC (Pure Appl. Chem., vol. 80, No. 1, p. 59) using glycine with carbonyl peak at 175.67 ppm as secondary standard.

### Reference Example 3: Determination of Water Uptake

Water adsorption/desorption isotherms were performed on a VTI SA instrument from TA instruments following a step-isotherm program. The experiment consisted in one or more runs performed on a sample material placed on the microbalance pan inside the instrument. Before measurement was started, the residual moisture content of the sample was removed by heating the sample to 100° C. (5 K/min heat ramp) and maintaining the sample under a nitrogen stream for 6 h. After the drying program, the temperature in the cell was lowered to 25° C. and kept isothermal during measurement. The microbalance was calibrated, and the weight of the dried sample was balanced (0.01 wt % maximum deviation in mass). Water uptake by the sample was measured as the increased weight over the dry sample. An adsorption curve was measured first by increasing the relative humidity (expressed as wt % of water in the atmosphere of the cell) to which the sample was exposed, and measuring the water uptake of the sample as the equalizing weight. The relative humidity was increased from 5 wt % to 85 wt % in 10 wt % increments, with the system policing the relative humidity for each increment, and monitoring the sample weight until attainment of equilibrium conditions after the sample was exposed to 85 wt % to 5 wt % relative humidity in increments of 10 wt % and the change in the weight of the sample (the water uptake) had been monitored and recorded.

### Reference Example 4: Measurement of Infrared Spectra

FT-IR (Fourier transform infrared) measurements were performed on a Nicolet 6700 spectrometer. The powdered material was compressed into a self-supporting pellet without the use of any additives. The pellet was introduced into a high vacuum cell (HV) accommodated in the FT-IR instrument. Measurement of the sample was preceded by preheating in high vacuum (10⁻⁵ mbar) at 300° C. for 3 h. Spectra were recorded after cooling the cell back down to 50° C. Spectra were recorded in the range from 4000 to 800 cm⁻¹ at a resolution of 2 cm⁻¹. The spectra obtained were depicted in a diagram having the wavelength (cm⁻¹) on the x-axis and the absorption (in arbitrary units “a.u.”). A baseline correction was carried out to quantitatively determine the peak heights and the ratios between these peaks. Changes in the range from 3000 to 3900 cm⁻¹ were analyzed and the band at 1880±5 cm⁻¹ was used as reference to compare two or more samples.

### Reference Example 5: Measurement of x-Ray Diffraction Spectra

The x-ray diffraction spectrum was recorded using a D8 Advance Series 2 from Bruker/AXS, which was equipped with a multiple sample changer.

### Example 1: Preparing an MWW Framework Structure Zeolite Comprising Boron and Titanium

Deionized water (841.82 g) in a glass beaker was admixed with piperidine (200 g), and the resulting mixture was stirred at room temperature for 5 min. Boric acid (203.8 g) was then admixed to the mixture and dissolved for 20 min, followed by a solution of tetrabutyl orthotitanate (17.75 g) dissolved in piperidine (99.24 g) admixed under agitation at a stirrer speed of 70 rpm, and the resulting mixture was stirred at room temperature for 30 min. The mixture was admixed with fumed silica (Cab-O-Sil® M7D, 147.9 g) under agitation, and the resulting mixture was stirred at room temperature for 1.5 h. The mixture had a pH of 11.3. The mixture was transferred into a 2.5 l autoclave and slowly heated to 170° C. over 10 hours at a heating rate of about 0.2 K/min and then was maintained at 170° C. for 160 h under agitation at a stirrer speed of 100 rpm. The pressure during the reaction was in the range from 8.3 to 9 bar. The suspension obtained had a pH of 11.2. The suspension was filtered and the filtercake was washed with deionized water until the wash liquor had a pH of less than 10. The filtercake was placed in a drying oven and dried at 120° C. for 48 h, heated to a temperature of 650° C. at a heating rate of 2 K/min and calcined at 650° C. in an air atmosphere for 10 h to obtain a colorless powder (101.3 g). The powder had a boron content of 1.3 wt %, reckoned as elemental boron, a titanium content of 1.3% by weight, reckoned as elemental titanium, and a silicon content of 40% by weight, reckoned as elemental silicon. The hydrocarbon content totaled 0.1% by weight. The water uptake determined as per Reference Example 3 was 13.7% by weight. The ¹¹B solid state NMR spectrum of the zeolitic material is shown in FIG. 1. The ²⁹Si solid state NMR spectrum of the zeolitic material is shown in FIG. 2. The FT-IR spectrum of the zeolitic material is shown in FIG. 3. The x-ray diffraction spectrum of the zeolitic material is shown in FIG. 4. The x-ray diffraction spectrum of the zeolitic material further has the following characteristics:

| Angle | d-Value | Intensity | Intensity |
|---|---|---|---|
| 2-Theta ° | Angstrom | Cps | % |
| 7.013 | 12.59532 | 274 | 20.3 |
| 7.238 | 12.20307 | 468 | 34.7 |
| 8.058 | 10.96352 | 326 | 24.2 |
| 10.137 | 8.71933 | 362 | 26.9 |
| 13.007 | 6.80071 | 134 | 10 |
| 14.278 | 6.19805 | 313 | 23.2 |
| 14.476 | 6.11373 | 408 | 30.3 |
| 14.924 | 5.93142 | 209 | 15.5 |
| 16.143 | 5.48597 | 249 | 18.5 |
| 18.048 | 4.91111 | 119 | 8.8 |
| 19.22 | 4.61418 | 192 | 14.3 |
| 20.448 | 4.33988 | 313 | 23.3 |
| 21.418 | 4.14544 | 239 | 17.8 |
| 21.857 | 4.06307 | 403 | 29.9 |
| 22.142 | 4.01139 | 424 | 31.5 |
| 22.687 | 3.91637 | 413 | 30.7 |
| 22.974 | 3.8681 | 747 | 55.5 |
| 23.97 | 3.70954 | 539 | 40 |
| 25.277 | 3.5206 | 446 | 33.1 |
| 26.321 | 3.38327 | 1346 | 100 |
| 27.289 | 3.26537 | 498 | 37 |
| 28.094 | 3.17361 | 511 | 37.9 |
| 28.947 | 3.08203 | 357 | 26.5 |
| 30.059 | 2.97051 | 210 | 15.6 |
| 32.004 | 2.79431 | 217 | 16.1 |
| 32.669 | 2.73889 | 229 | 17 |
| 33.757 | 2.65307 | 319 | 23.7 |
| 34.829 | 2.57383 | 233 | 17.3 |
| 36.837 | 2.43798 | 209 | 15.5 |
| 37.535 | 2.39427 | 197 | 14.6 |
| 38.316 | 2.34724 | 240 | 17.8 |
| 41.141 | 2.19233 | 204 | 15.2 |
| 41.982 | 2.15035 | 205 | 15.2 |
| 43.358 | 2.08522 | 213 | 15.8 |
| 45.169 | 2.00577 | 255 | 19 |
| 46.654 | 1.94533 | 266 | 19.8 |
| 46.968 | 1.93304 | 284 | 21.1 |
| 48.908 | 1.86079 | 255 | 19 |
| 49.494 | 1.84015 | 259 | 19.2 |
| 49.992 | 1.82295 | 232 | 17.2 |
| 51.38 | 1.77694 | 246 | 18.3 |
| 52.019 | 1.75659 | 255 | 18.9 |
| 53.699 | 1.70552 | 240 | 17.8 |
| 54.686 | 1.67707 | 230 | 17.1 |
| 57.016 | 1.61393 | 233 | 17.3 |
| 57.764 | 1.5948 | 225 | 16.7 |
| 58.87 | 1.56745 | 240 | 17.8 |
| 60.68 | 1.52495 | 248 | 18.4 |
| 62.067 | 1.49415 | 261 | 19.4 |
| 63.043 | 1.47336 | 262 | 19.5 |
| 65.449 | 1.42489 | 286 | 21.2 |
| 66.425 | 1.40631 | 348 | 25.9 |

### Example 2: Preparing a Pd/C Catalyst

A Pd-containing solution was prepared as follows: 15.80 g of Pd(NOₐ)₂ solution having a Pd content of 11% by weight was made up with completely ion-free water to an overall volume of 136 mL. 172 g of Supersorbon® activated carbon (particle size 0.7-1.0 mm) were saturated in this solution and then dried in a drying cabinet at 80° C. for 16 h. The catalyst was then calcined in a rotary tube oven at 400° C. for 4 h under flowing N₂. The 1% by weight Pd/C catalyst was subsequently doped with KOH. To this end, 24.89 g of a 5% by weight KOH solution were made up to 64 mL with completely ion-free water. 80.90 g of the 1% by weight Pd/C catalyst were saturated in this solution. The catalyst was subsequently dried in a drying cabinet at 80° C. for 16 h.

### Example 3: Synthesis of 2-methoxycyclohexanol (a Compound of Formula (I)) from Cyclohexene

In a reaction vessel, 1.00 g of the zeolitic material obtained as per Example 1 was mixed with 3.92 g of cyclohexene in 20 ml of methanol. The resulting mixture was admixed with 1.00 g of an aqueous hydrogen peroxide solution (50 wt % hydrogen peroxide). The mixture was stirred under reflux for 4 h. After filtering off the catalyst and weighing back the filtrate thus obtained, a sample was taken of the filtrate. The H₂O₂ content of this sample was determined cerimetrically. The filtrate was quenched with sodium sulfite in order to decompose any H₂O₂ still present. A sample was then taken in order to use gas chromatography (GC) analysis to determine the molar amounts of compounds (I) to (Id), which potentially form as per the reaction scheme shown below. The molar amounts were used to compute the selectivity of the catalyst as the molar amount of the desired product in the form of 2-methoxycyclohexanol (compound of formula (I)), based on the molar amount of the mixture of compounds of formulae (I) to (Id). The result was a selectivity of 93%. After separating off the organic phase, distillation gave the 2-methoxycyclohexanol in a purity of greater than 95%. 2-Methoxycyclohexanol aside the mixture obtained was essentially comprised of dihydroxycyclohexane. The following conditions were used for the distillation: external temperature: 100 to 102° C.; pot temperature: 90 to 92° C. (column trace heating: 90° C.); overhead temperature: 81 to 82° C.; overhead pressure: 20.0 mbar.

### Example 4: Synthesis of Guaiacol (a Compound of Formula (II)) from 2-methoxycyclohexanol

A reaction column was filled with 13 mL (5 g) of catalyst (1% by weight of Pd on activated carbon, prepared as per Example 2 and prewashed with 5% by weight aqueous KOH solution) and packed with quartz rings (30 mL below the catalyst bed, 30 ml above the catalyst bed). The catalyst was activated by flushing with a gas mixture N₂/H₂ (95:5) at 400° C. for 15 min. This was followed by flushing with H₂ for a further 15 min. After purging with N₂, the reactor temperature was adjusted to 300° C. (what is referred to as the reactor temperature is the temperature of the fixed catalyst bed; this temperature is measured via a thermocouple arranged radially and longitudinally in the center of the fixed catalyst bed). An additional vaporizer was attached to prevaporize the 2-methoxycyclohexanol feed stream from Example 3 at 275° C. The carrier gas used was N₂ (20 L/h) and H₂ (2.5 L/h). The 2-methoxycyclohexanol obtained as per Example 3 was then introduced as feed stream (25% by weight aqueous solution) into the reactor system (prevaporizer+reactor) at a flow rate of 6 g/h. Following 100 h of continuous operation, a 2-phase product mixture was obtained. The 2-phase product mixture thus obtained was subjected to a distillation to recover the guaiacol as product (conversion rate: 52%, (based on 2-methoxycyclohexanol used); selectivity: 72% (based on converted 2-methoxycyclohexanol)). The following conditions were used for the distillation: external temperature: 72 to 78° C.; pot temperature: 60 to 72° C.; overhead temperature: 41 to 50° C.; overhead pressure: 1.5 mbar.

### Example 5: Synthesis of Vanillin (a Compound of Formula (IV)) from Guaiacol Via a Mandelic Acid Derivative (a Compound of Formula (III)) as Intermediate

Glyoxylic acid (23.74 g, 50% by weight in H₂O, 0.16 mol) and 7% by weight NaOH (91.6 g, 0.16 mol) were introduced into a reaction vessel and stirred at room temperature. Following the slight exothermism of the mixing process, a mixture of guaiacol as per Example 4 (46.5 g, 0.2 mol) and 10% by weight aqueous NaOH (80.1 g, 0.2 mol) was added in the course of 45 min. The mixture was subsequently stirred at room temperature for 4.5 h. After this period, the solution obtained was homogeneous. The solution obtained had a pH of about 11 and was then carefully acidified with concentrated H₂SO₄ (10.9 g, 0.11 mol) until the pH had a value of 4.5. Then, the mixture was extracted with toluene (3×300 mL) in order to remove, and recycle, unconverted guaiacol. The aqueous phase obtained was further acidified with 97% to 99% by weight H₂SO₄ (15.4 g, 0.15 mol) until the pH had reached a value of about 1.0. An aqueous solution of the mandelic acid derivative of formula (III) was obtained. This crude solution of the mandelic acid derivative was heated together with toluene (160 mL) to a temperature of 90° C. Then, an aqueous 20% by weight FeCl₃ solution (282.4 g) was admixed over 30 min, causing the red solution to turn black. The mixture was then further stirred at 90° C. for 2 h. Toluene (90 mL) was then admixed at 90° C. and the batch was cooled down to room temperature. The two phases were separated and the aqueous phase was additionally extracted with toluene (150 mL) five times. The combined organic phases were washed with water (350 mL) and then concentrated under reduced pressure to obtain vanillin of the formula of a compound of formula (IV) as a slightly beige solid (13.6 g, selectivity 64% based on glyoxylic acid).

### SHORT DESCRIPTION OF FIGURES

FIG. 1 shows the ¹¹B solid state NMR spectrum of the zeolite according to Example 1, as measured according to Reference Example 1. The ¹¹B chemical shift (in ppm) is shown on the x-axis, while the intensity (*10⁶) is shown on the y-axis. The scale divisions on the x-axis are, from left to right, at 40, 20, 0, −20. The scale divisions on the y-axis are, from bottom to top, at 0, 1, 2, 3, 4.

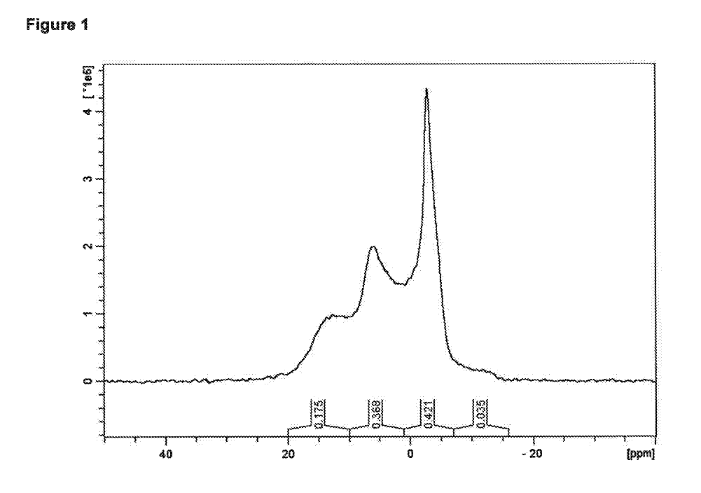

FIG. 2 shows the ²⁹Si solid state NMR spectrum of the zeolite according to Example 1, as measured according to Reference Example 2. The ²⁹Si chemical shift (in ppm) is shown on the x-axis, while the intensity (*10⁶) is shown on the y-axis. The scale divisions on the x-axis are, from left to right, at −90, −100, −110, −120, −130. The scale divisions on the y-axis are, from bottom to top, at 0, 20, 40, 60, 80, 100.

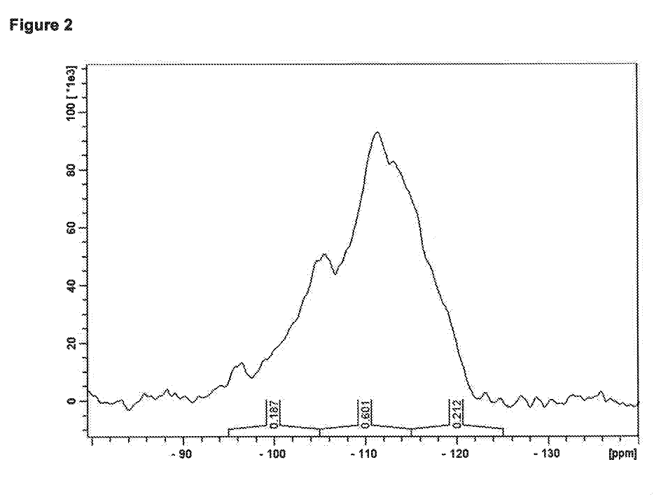

FIG. 3 shows the FT-IR spectrum of the zeolite according to Example 1, as measured according to Reference Example 4. The wavelength (in cm⁻¹) is shown on the x-axis and the extinction is shown on the y-axis. The scale divisions on the x-axis are, from left to right, at 4000, 3500, 3000, 2500, 2000, 1500. The scale divisions on the y-axis are, from bottom to top, at 0.00, 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, The wavenumbers indicated on the individual peaks in cm⁻¹ are, from left to right, 3748, 3719, 3689, 3623, 3601, 3536, 1872.

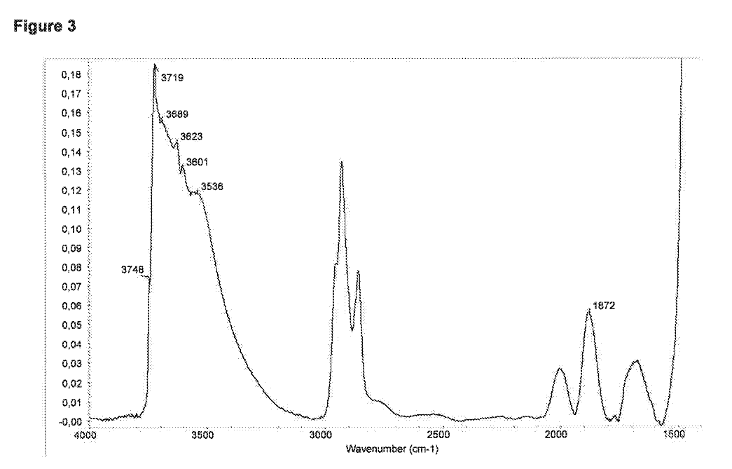

FIG. 4 shows the x-ray diffraction pattern (copper K-alpha radiation) of the zeolite according to Example 1, measured according to Reference Example 5. The degree values (2 theta) are shown on the x-axis and the intensity (Lin (counts)) are shown on the y-axis. The scale divisions on the x-axis are, from left to right, at 2, 10, 20, 30, 40, 50, 60, and 70. The scale divisions on the y-axis are, from bottom to top, at 0 and 3557

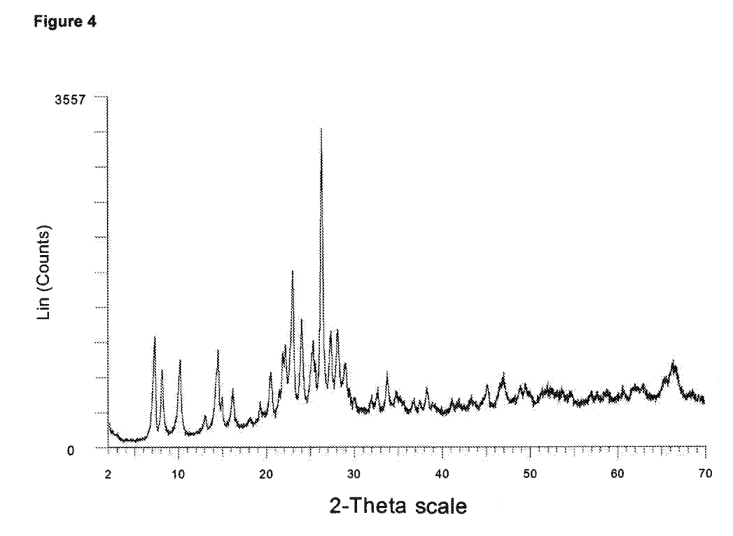

### LITERATURE CITED

GB 2 252 556 A
A. Corma et al., “Activity of Ti-Beta Catalyst for the Selective Oxidation of Alkenes and Alkanes”, Journal of Catalyis (1994), vol. 145, pp. 151-158
Y. Goa et al., “Catalytic Performance of [Ti, Al]-Beta in the Alkene Epoxidation Controlled by the Postsynthetic Ion Exchange”, Journal of Physical Chemistry B 2004, vol. 108, pp. 8401-8411
E. G. Derouane et al., “Titanium-substituted zeolite beta: an efficient catalyst in the oxy-functionalisation of cyclic alkenes using hydrogen peroxide in organic solvents”, New. J. Chem., 1998, pp. 797-799
M. A. Uguina et al., J. Chem. Soc., Chem. Commun., 1994, p. 27

1-15. (canceled)

16. A method of preparing a compound of formula (IV) 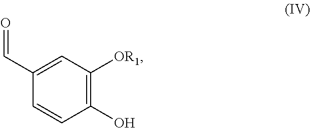
wherein R₁ is alkyl of 1 to 4 carbon atoms, comprising:
(i) providing a liquid mixture comprising cyclohexene, an alcohol R₁OH, hydrogen peroxide and optionally a solvent;
(ii) reacting the cyclohexene with the hydrogen peroxide and the alcohol R₁OH in the mixture provided as per (i) in the presence of a catalyst comprising a zeolite of framework structure MWW to obtain a mixture comprising the compound of formula (I) 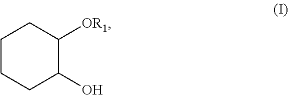
wherein the framework of the zeolite as per (ii) comprises silicon, titanium, boron, oxygen and hydrogen;
(iii) separating the compound of formula (I) off from the mixture obtained as per (ii) to obtain a mixture concentrated in respect of the compound of formula (I);
(iv) dehydrogenating the formula (I) compound present in the concentrated mixture obtained as per (iii) to obtain a mixture comprising a compound of formula (II) 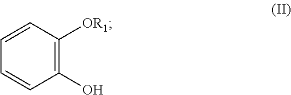
(v) formylating the formula (II) compound present in the mixture obtained as per (iv) to obtain a mixture comprising the compound of formula (IV).

17. The method according to claim 16, wherein R₁ is methyl or ethyl, preferably methyl.

18. The method according to claim 16, wherein the liquid mixture provided as per (i) has a molar ratio prior to the reaction as per (ii) of cyclohexene:R₁OH in the range from 1:1 to 1:50, preferably from 1:3 to 1:30 and more preferably from 1:5 to 1:10, and a molar ratio prior to the reaction as per (ii) of cyclohexene:hydrogen peroxide in the range from 1:1 to 5:1, preferably from 1.5:1 to 4.5:1, more preferably from 2:1 to 4:1.

19. The method according to claim 16, wherein the liquid mixture provided as per (i) comprises no solvent.

20. The method according to claim 16, wherein the reaction as per (ii) is carried out at a temperature in the range from 40 to 150° C., preferably from 50 to 125° C., more preferably from 70 to 100° C., wherein the duration of the reaction as per (ii) is preferably in the range from 1 to 12 h, more preferably from 1.5 to 10 h, more preferably from 2 to 8 h, and wherein the mass ratio of hydrogen peroxide:zeolite of framework structure MWW at the start of the reaction as per (ii) is preferably in the range from 10:1 to 0.1:1, more preferably from 1:1 to 0.2:1, more preferably from 0.75:1 to 0.25:1.

21. The method according to claim 16, wherein not less than 99 wt %, preferably not less than 99.5 wt %, more preferably not less than 99.9 wt % of the framework of the zeolite as per (ii) consists of silicon, titanium, boron, oxygen and hydrogen, and wherein, in the zeolite of framework structure MWW, the molar ratio of B:Si is preferably in the range from 0.02:1 to 0.5:1, more preferably from 0.05:1 to 0.15:1, and the molar ratio of Ti:Si is preferably in the range from 0.01:1 to 0.05:1, more preferably from 0.017:1 to 0.025:1, while the zeolite of framework structure MWW is preferably obtained as per a method comprising:
(a) providing an aqueous synthesis mixture comprising a silicon source, a boron source, a titanium source and an MWW-templating compound, wherein the temperature of the aqueous synthesis mixture is not more than 50° C.;
(b) heating the aqueous synthesis mixture provided as per (a) from the temperature of not more than 50° C. to a temperature in the range from 160 to 190° C. in the course of a period of at most 24 h;
(c) subjecting the synthesis mixture as per (b) to hydrothermal synthesis conditions under autogenous pressure in a closed system at a temperature in the range from 160 to 190° C. to obtain a precursor to the zeolite of framework structure MWW in its mother liquor;
(d) separating the precursor to the zeolite of framework structure MWW off from its mother liquor; and
(e) calcining the MWW framework structure zeolite precursor separated off as per (d) to obtain the zeolite of framework structure MWW.

22. The method according to claim 16, wherein the mole percentage for the compound of formula (I) in the mixture obtained from the reaction as per (ii), based on the sum total of mole percentages for the compounds of formulae (I), (Ib), (Ic), (Id) and (Ie) 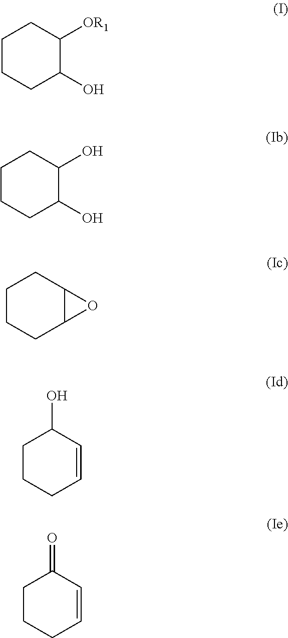
in the mixture obtained from the reaction as per (ii) is not less than 85%, preferably not less than 90%.

23. The method according to claim 16, wherein as per (iii) the step of separating the compound of formula (I) off from the mixture obtained as per (ii) comprises a distillation, wherein the mixture obtained from the distillation and concentrated in respect of the compound of formula (I) comprises the compound of formula (I) at preferably not less than 90 wt %, more preferably at not less than 95 wt %, more preferably at more than 95 wt %.

24. The method according to claim 16, wherein the mixture obtained as per (iii), concentrated in respect of the compound of formula (I), is vaporized, preferably at a temperature in the range from 175 to 375° C., more preferably from 225 to 325° C., more preferably from 250 to 300° C., prior to the dehydrogenation as per (iv).

25. The method according to claim 16, wherein the dehydrogenation as per (iv) is carried out in the presence of a heterogeneous catalyst, wherein the catalyst preferably comprises a noble metal selected from the group consisting of Pd, Rh, Pt and a combination of two or more thereof, wherein the noble metal is preferably supported by one or more than one carrier material, which is preferably selected from the group consisting of activated carbon, aluminum oxide, silicon oxide and a combination of two or more thereof, wherein the catalyst more preferably comprises palladium as noble metal and activated carbon as carrier material, and wherein the dehydrogenation as per (iv) is carried out at a catalyst temperature, preferably, in the range from 200 to 400° C., more preferably from 250 to 350° C., more preferably from 275 to 325° C.

26. The method according to claim 16, wherein the formylating step as per (v) is preceded by the compound of formula (II) being separated off from the mixture obtained from (iv) to obtain a mixture concentrated in respect of the compound of formula (II), wherein the step of separating off preferably comprises a distillation, wherein the mixture obtained, which is concentrated in respect of the compound of formula (II), comprises the compound of formula (II) at preferably not less than 95 wt %, more preferably at not less than 98 wt %.

27. The method according to claim 16, wherein the formylating step as per (v) comprises:
(v-1) reacting the formula (II) compound present in the mixture obtained as per (iv) with glyoxylic acid OHC—COOH, preferably in aqueous phase, to obtain a mixture comprising a compound of formula (III) 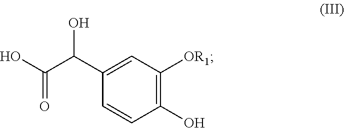
(v-2) preferentially purifying the mixture obtained as per (v-1) in respect of the compound of formula (III) to obtain a preferably aqueous mixture comprising the compound of formula (III), wherein the step of purifying preferably comprises an extraction;
(v-3) oxidatively decarboxylating the formula (III) compound present in the mixture obtained as per (v-1), preferably as per (v-2), to obtain a mixture comprising the compound of formula (IV).

28. The method according to claim 27, wherein the step of reacting as per (v-1) is carried out in the presence of a Bronstedt base, preferably selected from the group consisting of alkali metal hydroxides, alkaline earth metal hydroxides and a mixture of two or more thereof, more preferably from the group consisting of alkali metal hydroxides and a mixture of two or more thereof, more preferably in the presence of sodium hydroxide, wherein the step of reacting as per (v-1) is carried out at a temperature preferably in the range from 10 to 40° C., more preferably in the range from 15 to 35° C., more preferably in the range from 20 to 30° C.

29. The method according to claim 27, wherein the step of oxidatively decarboxylating as per (v-3) is carried out in the presence of an oxidizing agent selected from the group consisting of CuO, PbO₂, MnO₂, Co₃O₄, HgO, Ag₂O, Cu(II) salts, Hg(II) salts, Fe(III) salts, Ni(III) salts, Co(III) salts, chlorates and a mixture of two or more thereof, preferably from the group consisting of CuO, MnO₂, Cu(II) salts, Fe(III) salts and a mixture of two or more thereof, more preferably in the presence of FeCl₃, wherein the step of oxidatively decarboxylating as per (v-3) is carried out at a temperature preferably in the range from 60 to 110° C., more preferably from 70 to 100° C., more preferably from 80 to 95° C.

30. The method according to claim 16, comprising:
(vi) separating the compound of formula (IV) off from the mixture obtained as per (v), preferably from the mixture obtained as per (v-3);
wherein the mixture obtained as per (v), preferably as per (v-3), preferably comprises water and one or more than one organic solvent, and wherein (vi) preferably comprises:
(vi-1) separating the organic phase from the aqueous phase to obtain an organic phase comprising the compound of formula (IV);
(vi-2) optionally extracting the aqueous phase with one or more than one organic solvent to obtain one or more than one further organic phase comprising the compound of formula (IV);
(vi-3) preferably washing the organic phase or phases comprising the compound of formula (IV) and obtained as per (vi-1) or, respectively, as per (vi-1) and (vi-2);
(vi-4) concentrating the organic phase obtained as per (vi-1) or the organic phases obtained as per (vi-1) and (vi-2), preferably the organic phase washed as per (vi-3) or the organic phase washed as per (vi-3), in respect of the compound of formula (IV), preferably under reduced pressure compared with ambient pressure.
